(19) United States
(12) Patent Application Publication
(10) Pub. No.: US 2018/0045735 A1
(43) Pub. Date: Feb. 15, 2018

(54) Methods and Compositions For The Diagnosis And Treatment Of Cancer and Autoimmune Disorders

(71) Applicant: Immport Therapeutics, Inc., Irvine, CA (US)

(72) Inventors: Xiaowu Liang, La Jolla, CA (US); Douglas Molina, Irvine, CA (US); William Morrow, Irvine, CA (US)

(21) Appl. No.: 15/446,961

(22) Filed: Mar. 1, 2017

Related U.S. Application Data
(63) Continuation of application No. 13/820,464, filed on Aug. 6, 2013, now abandoned.
(60) Provisional application No. 61/380,063, filed on Sep. 3, 2010.

(51) Int. Cl. G01N 33/68; G01N 33/574; G01N 33/564

(52) U.S. Cl. CPC G01N 33/6854; G01N 33/564; G01N 33/57415

(57) ABSTRACT

Compositions, devices, and methods are contemplated for predicting a patient's likelihood of having a disease. An antigen composition can have a plurality of autoantibody reactive antigens associated with a carrier, where at least two of the antigens have quantified and known relative autoantibody reactivities with respect to sera of a population affected by a disease. The at least two antigens can also have a known association with a disease parameter. A method can include determining autoantibody reactivity against one or more antigens or their variants in a serum sample obtained from a patient, where the autoantibody reactivity against one or more of the antigens indicates an increased likelihood of the patient having a disease.

This application is a continuation application of U.S. application Ser. No. 13/820,464 filed Mar. 1, 2013, which is a U.S. national phase filing of International Application No. PCT/US11/50210 filed Sep. 1, 2011, which claims the benefit of priority to U.S. provisional application having Ser. No. 61/380,063 filed on Sep. 3, 2010. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is compositions and methods for diagnosis and treatment of various disorders and diseases.

### BACKGROUND

Currently, no diagnostic test, therapeutic treatment, or vaccine has been developed as a result of high-throughput proteomic research methodologies. This seems surprising, as a solid foundation for such advances was seemingly laid during the recent genomics era.

Efforts spurred on by the Human Genome Project (HGP), whose goal was to sequence the human genome, resulted in detailed genome sequencing and annotation of infectious agents such as viruses, bacteria, and parasitic eukaryotes. The information that became available included the identification and sequences of all the open reading frames (ORFs), which are the instruction set for the assembly of proteins. The ORFs can be translated into an intermediary set of instructions called messenger RNA (mRNA), which can then be translated into proteins. Equipped with this information, researchers could determine which proteins were expressed in which tissue by measuring mRNA expression via oligos designed using the sequence information as probes using a Northern blot.

Unfortunately, traditional Northern blots became a bottleneck for researchers who desired to look at the expression of many genes. The need to multiplex northern blots lead to tools and methodologies that would allow for multiple parallel experiments, or multiplexing, to be performed. A significant advance was the creation of microarray printers, robotic devices that move in the X, Y, and Z directions and deposits tiny volumes of probes, or reporters, in an organized fashion onto a surface, generally a microscope slide. This methodology allowed for the creation of high density and highly multiplexed Northern blots. To gather data from these microscopic Northern blots, confocal microscope-based fluorescence scanners were used. These efforts were coupled with tools designed to analyze the significant amount of generated data. Such advances allowed researchers to collect massive amounts of data about the expression profiles of normal and diseased tissues.

Much effort has been exerted to connect the descriptive sequencing data and expression profiling data noted above to the prediction, or treatment, of disease. While there remains hope that such efforts will lead to valuable insights into human diseases, knowing identity and relative abundance does not seem to be sufficiently useful.

Surprisingly, the inventors have found that it is actually functional data that is required to accurately survey immune responses to human diseases. However, no algorithm is known to the inventors that can predict significantly antigenic epitopes from foreign proteins (e.g., bacteria, parasites, etc.), let alone endogenous, human proteins. Similarly the abundance or changes in the abundance of proteins in the body has not been a useful predictor of disease. Recently, it has been shown that circulating autoantibodies might be useful for predicting disease. Instead, circulating autoantibodies must be measured to determine which autoantibodies might serve as predictors of disease. However, the methodologies known to the inventors are unable to create a large enough expressible library of human proteins to cast a wide net, and to express and screen these proteins in a high-throughput manner. Current practice in the art teaches that for one to accurately detect autoantibodies, the protein(s) being used as bait for the antibodies should retain most, or all, of the post-translational modifications that would be present on the protein as it is naturally expressed in the body. Another concept common in the art is that researchers assume that there is a need to purify proteins before using them in functional assays, a process which may take months, to even years, for a single protein.

Consequently, there remains a large, unmet need to provide improved compositions and methods of antigen and autoantibody detection and monitoring for diagnostic and therapeutic applications.

### SUMMARY OF THE INVENTION

Based on the above noted difficulties, a proteomics approach aiming to profile human autoantibodies reactivity that uses unpurified proteins expressed in an E. coli based cell-free system was not expected by those practicing the art to prove useful. Surprisingly, however, the inventors found that such approach worked very well, and could be used to identify both well-known and novel antigens and autoantibodies that could have not been identified using conventional methodologies.

The inventive subject matter discussed herein provides apparatus, systems and methods for identifying, analyzing, and monitoring autoantibody reactivity to specific antigens or sets of antigens, which can have diagnostic, prognostic, and therapeutic value, specifically with respect to various human diseases. This is especially important in the diagnosis and/or treatment of various human diseases, cancers, and autoimmune disorders. Exemplary diseases include breast cancer, lupus, lupus nepritis, systemic lupus erythematosus, polymyositis, rheumatoid arthritis, scleroderma, and Sjögren's syndrome, although the specific disease will depend upon the specific antigens or sets of antigens.

Thus, in some aspects, the disease is breast cancer, and the set of antigens has a sequence according to one or more of GENE ID BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, or UTP14a, or fragments thereof, or the disease is lupus nephritis (LN), and the set of antigens has a sequence according to one or more of GENE ID CD1D, IL6R, IRF8, ITGA2B, MYO1A, MYO7B, PSG1, PTBP1, or TPO, or fragments thereof. In further aspects, the disease is systemic lupus erythematosus (SLE), and the set of antigens has a sequence according to one or more of GENE ID CD1C, CD46, CENPQ, CFB, DPP4, HLA-DQB1, IL6R, ITGB2, KRTAP9-3, MLF1IP, MYT1L, POLR2H, SLC7A5, or TPO, or fragments thereof, or the disease is Lupus (SLE+LN), and the set of antigens has a sequence according to one or more of GENE ID DPP4, IL6R, ITGB2, MLF1IP, MYO1A, POLR2H, or TPO, or fragments thereof, or the disease is polymyositis (P), and the set of antigens has a sequence according to one or more of GENE ID CD14, CD1C, CD46, CD55, CFB, COL9A1, COLQ, DLAT, DPP4, FGF7, H3F3B, IL1RAPL2, IL6R, IL8, ITGB2, KRTAP9-3, MLF1IP, MYT1L, PADI4, PIP4K2C, PLAUR, POLR2H, POLR2I, PSG1, SLC7A5, or STK19, or fragments thereof. In yet further aspects, the disease is rheumatoid arthritis (RA), and the set of antigens has a sequence according to one or more of GENE ID APOH, BANK1, BLK, CD1C, CD14, CD3E, CD70, CD80, CD86, CEACAM6, CEACAM8, CENPT, CFB, COL1A2, DDC, DPP4, FCGR1A, H2AFX, H2AFY, H3F3B, HBA1, HBA2, HBD, HLA-DQB1, HSP90B1, HSPB7, IGHG2, IGHG4, IGHM, IGHV4-31, IL12A, IL6, IL6R, ITGB3BP, KRTAP13-1, KRTAP9-3, MBP, MLF1IP, MOBP, MS4A8B, MYH9, MYO1D, MYT1L, NMNAT2, NOL1, PDCD1, PIP4K2C, POLR2C, POLR2H, POLR2I, POLR2J2, POLR3D, PSIP1, SRP19, STAT4, or STK19, or fragments thereof, or the disease is Scleroderma (Sc), and the set of antigens has a sequence according to GENE ID IL6R, or a fragment thereof, or the disease is Sjögren's Syndrome (Sj), and the set of antigens has a sequence according to one or more of GENE ID APOH, CALR3, CD1C, CD14, CD34, CD3E, CD46, CD69, CD93, CEACAM8, CENPA, CENPQ, CFB, CHRNA1, COL20A1, COL4A6, DPP4, FCGR3A, H1F0, H2AFX, H3F3B, HBA1, HBA2, HBD, HBM, HLA-C, HLA-DQB1, HLA-F, HSPB7, IFNG, IGFL2, IGH2, IGHV7-81, IL1RAPL2, IL6R, ITGB2, KRT73, KRT19, KRTAP9-3, KRTAP9-8, MBP, MLF1IP, MYT1L, NOLA3, POLR2H, POLR2I, POLR3D, POLR3H, PTBP1, STK19, or UEVLD, or fragments thereof.

In one aspect, the inventive subject matter provides a new and useful tool that can accurately survey human diseases via the multiplexed combination of unpurified E. coli expressed proteomes, autoantibody detection, and characterized sera samples from human disease populations.

In another aspect, an antigen composition has a plurality of autoantibody reactive antigens associated with a carrier. At least two of the antigens can have (a) quantified and known relative autoantibody reactivities with respect to sera of a population affected by a disease, and (b) a known association with a disease parameter. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It is contemplated that the known reactivities may be characterized by a variety of factors, however, it is particularly preferred that the known reactivities are characterized by strength of immunogenicity and/or time course of the infection. It is generally preferred that the parameter is activity state of the disease, a previous exposure to the pathogen, the duration of exposure to the pathogen, a chronic infection, past disease, active infection, inactive infection, at least partial immunity to infection with the pathogen, and/or outcome upon treatment.

In yet another aspect, a method of predicting a likelihood of a patient having a disease or detecting a disease in a patient is contemplated, which includes the step of determining autoantibody reactivity against one or more antigens, or their variants, in a serum sample obtained from a patient. The presence of autoantibody reactivity against one or more of the antigens can advantageously indicate an increased likelihood of the patient having a disease.

In another embodiment, a method of predicting a likelihood of a patient having a disease can include determining autoantibody reactivity against one or more antigens, or their variants, in a sera sample obtained from a patient. A likelihood of a disease can then be predicted from reference samples derived from sera of patients diagnosed as having the disease, such that increased or decreased autoantibody reactivity against selected antigens can be positively correlated with increased likelihood of a disease in the patient.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 shows representative images of a Tumor Associated Antigen (TAA) chip probed with serum from breast cancer patients and controls, showing several proteins that are recognized by antibodies in the serum in panels 1-2 and 5-8.

FIG. 2 is a histogram of the image data depicting the mean signal intensity for cancer patients (CA), population controls (P), and Bonferroni corrected p-value.

FIG. 3 shows representative images of a Tumor Associated Antigen (TAA) chip probed with serum from patients with cervical cancer (right panel) and a control group (left panel).

FIGS. 4-5 are representative images of a Human Autoimmunity (HA) chip probed with serum samples from patients with Sjögren's Syndrome, and serum samples from patients with Lupus, respectively.

FIG. 6 is a heat map of signal intensity data, and FIGS. 7A-7B is an enlarged view of a portion of the heat map of FIG. 2C.

FIG. 8 is a chart comparing the mean signal intensities and standard errors for normal/healthy sera (N), Sjögren's Syndrome patient sera (Sj), lupus nephritis patient sera (LN) and systemic lupus erythematosus (SLE).

FIG. 9 shows representative images of a HA chip probed with anti-HA high affinity rat monoclonal to verify expression of proteins.

FIG. 10 is a heat map of signal intensity data, and FIG. 11 is an enlarged view of a portion of the heat map of FIG. 3B.

FIG. 12 is a chart comparing the mean signal intensities and standard errors for normal/healthy sera (N), sera from lupus patients (L), and Benjamini-Hochberg corrected p-values (BHp).

FIG. 13 is a chart comparing the mean signal intensities and standard errors for normal/healthy sera (N), sera from lupus nephritis patients (LN), and Benj amini-Hochberg corrected p-values (BHp).

FIG. 14 is a chart comparing the mean signal intensities and standard errors for normal/healthy sera (N), sera from systemic lupus erythematosus patients (SLE), and Benjamini-Hochberg corrected p-values (BHp).

FIG. 15 is a chart comparing the mean signal intensities and standard errors for normal/healthy sera (N), sera from polymyositis patients (P), and Benjamini-Hochberg corrected p-values (BHp).

FIG. 16 is a chart comparing the mean signal intensities and standard errors for normal/healthy sera (N), sera from rheumatoid arthritis patients (RA), and Benjamini-Hochberg corrected p-values (BHp).

FIG. 17 is a chart comparing the mean signal intensities and standard errors for normal/healthy sera (N), sera from scleroderma patients (Sc), and Benjamini-Hochberg corrected p-values (BHp).

FIG. 18 is a chart comparing the mean signal intensities and standard errors for normal/healthy sera (N), sera from Sjögren's Syndrome patients (Sj), and Benjamini-Hochberg corrected p-values (BHp).

FIG. 19 is a heat map of signal intensity data of seven lupus nephritis patients.

FIGS. 20-26 are various charts of the serial bleeds from patient data.

FIGS. 27A-27H show a heat map of signal intensity data.

FIGS. 28-29 are charts comparing the signal difference in population controls (PC) or relative control (RC) as the baseline, respectively, versus cases (CS).

FIG. 30 is a representative image of a sub-array representing approximately 207 different expression products and 18 control spots visualized using the C-terminal HA tag and the anti-HA antibody.

FIG. 31 is a chart showing a distribution of mean signal intensities for the QC probing.

FIGS. 32-33 are charts showing the percentage of expression products recognized.

FIG. 34 is a heat map with the individual normal donors (rows) and the proteins (columns), and FIGS. 35A-35B are a histogram of mean signal intensities of the proteins.

FIG. 36 is a heat map showing the reactivity pattern of the 143 serum samples, and FIG. 37 is a histogram of all the reactive proteins.

FIG. 38 is a chart of the mean signal intensities, and FIG. 39 is a receiver operator curve using the proteins listed in FIG. 8C.

FIG. 40 is a heat map, and FIG. 41 is a histogram of mean signal intensities of the proteins.

FIG. 42 is a bar chart that compares reactivity of a lupus group with disease controls.

FIGS. 43-44 are flowcharts of various embodiments of methods of predicting a likelihood of a patient having a disease.

### DETAILED DESCRIPTION

One should appreciate that the disclosed techniques provide many advantageous technical effects including the ability to (a) identify biologically relevant antigens, sets of antigens, autoantibodies, and sets of autoantibodies, (b) enable the monitoring and analysis of treatment efficacy, via longitudinal monitoring of reactivity of an autoantibody, or a set of autoantibodies, against select human proteins, (c) identify, analyze, and monitor autoantibody reactivity to specific human protein antigens or antigen sets to facilitate diagnosis, prognosis, and treatment of cancers such as breast and pancreatic cancers or autoimmune disorders such as renal and non-renal lupus, polymyositis, rheumatoid arthritis, Scleroderma, and Sjögren's Syndrome, and (d) accurately survey human diseases via the combination of: unpurified proteomes, autoantibody detection and monitoring, and characterized sera samples, especially as they relate to use in diagnostic and therapeutic compositions and methods.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In the following description, antigens are identified by either the gene descriptor for the gene that encodes the protein antigen or the name of the protein antigen. Thus, it should be understood that where the context indicates that a sequence or antigen is a protein sequence, a gene name for that sequence or antigen denotes the protein product for that gene.

The inventors have discovered numerous antigens that are capable of triggering autoantibody reactivity from a variety of human diseases and disorders, including breast cancer, pancreatic cancer, renal lupus, non-renal lupus, polymyositis, rheumatoid arthritis, Scleroderma, and Sjögren's Syndrome. It is contemplated that such antigens can be used by themselves, or more preferably, in combination with other antigens in the manufacture of a diagnostic devices, therapeutic compositions, and vaccines.

Contemplated compositions, devices, and methods comprise autoantibody reactive antigens from various human diseases including, for example, breast cancer, pancreatic cancer, renal lupus, non-renal lupus, polymyositis, rheumatoid arthritis, Scleroderma, and Sjögren's Syndrome, which could be used as a vaccine, as diagnostic markers, or as therapeutic agents. In particularly preferred embodiments, the antigens have quantified and known relative reactivities with respect to sera of a population infected with a disease, and have a known association with a parameter of the disease. Thus, the specific antigens can have a statistically high probability to elicit autoantibody responses in a relatively large group of patients.

In one embodiment, an antigen composition can include a plurality of autoantibody reactive antigens associated with a carrier. The antigens are preferably selected from the group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, UTP14a, DPP4, IL6R, ITGB2, MLF1IP, MYO1A, POLR2H, CD1D, IRF8, ITGA2B, MYO7B, PSG1, PTBP1, CD1C, CD46, CENPQ, CFB, HLA-DQB1, KRTAP9-3, MYT1L, SLC7A5, TPO, CD14, CD55, COL9A1, COLQ, DLAT, FGF7, H3F3B, IL1RAPL2, IL8, PADI4, PIP4K2C, PLAUR, STK19, APOH, BANK1, BLK, CD3E, CD70, CD80, CD86, CEACAM6, CEACAM8, CENPT, COL1A2, DDC, FCGR1A, H2AFX, H2AFY, HBA1, HBA2, HBD, HSP90B1, HSPB7, IGHG2, IGHG4, IGHM, IGHV4-31, IL6, ITGB3BP, KRTAP13-1, MBP, MOBP, MS4A8B, MYH9, MYO1D, NMNAT2, NOL1, PDCD1, POLR2C, POLR2J2, POLR3D, PSIP1, SRP19, STAT4, CALR3, CD34, CD69, CD93, CENPA, CHRNA1, COL20A1, COL4A6, FCGR3A, H1F0, HBM, HLA-C, HLA-F, IFNG, IGFL2, IGH2, IGHV7-81, KRT73, KRT19, KRTAP9-8, NOLA3, POLR3H, and UEVLD, or fragments thereof. Additional information regarding each of the above-identified antigens is provided in Table 1 below.

TABLE 1

| | Current | | | | |
|---|---|---|---|---|---|
| Old | Gene | | | | UniGene |
| Symbol | Symbol | Gene Title | Class | Function | ID |
| B2G1 | APOH | apolipoprotein H (beta-2- | Serum protein | Carrier | Hs.445358 |
| | | glycoprotein I) | | protein | |
| BANK1 | BANK1 | B-cell scaffold protein | Signaling | Immune | Hs.480400 |
| | | with ankyrin repeats 1 | | | |
| BLK | BLK | B lymphoid tyrosine | Signaling | Immune | Hs.146591 |
| | | kinase | | | |
| CALR3 | CALR3 | calreticulin 3 | Chaperone | Protein | Hs.304020 |
| | | | | processing | |
| CD14 | CD14 | CD14 molecule | Receptor | Innate | Hs.163867 |
| | | | | immunity | |
| CD18 | ITGB2 | integrin, beta 2 | Cell adhesion | Cell-cell | Hs.375957 |
| | | (complement component 3 | | interaction | |
| | | receptor 3 and 4 subunit) | | | |
| CD1C | CD1C | CD1c molecule | Antigen | Immune | Hs.132448 |
| | | | presentation | | |
| CD1D | CD1D | CD1d molecule | Antigen | Immune | Hs.1799 |
| | | | presentation | | |
| CD26 | DPP4 | dipeptidyl-peptidase 4 | Protease | Protein | Hs.368912 |
| | | | | processing | |
| CD34 | CD34 | CD34 molecule | Cell adhesion | Immune | Hs.374990 |
| CD3E | CD3E | CD3e molecule, epsilon | Signaling | Immune | Hs.3003 |
| | | (CD3-TCR complex) | | | |
| CD46 | CD46 | CD46 molecule, | Complement | Innate | Hs.510402 |
| | | complement regulatory | | immunity | |
| | | protein | | | |
| CD55 | CD55 | CD55 molecule, decay | Complement | Innate | Hs.126517 |
| | | accelerating factor for | | immunity | |
| | | complement (Cromer | | | |
| | | blood group) | | | |
| CD64A | FCGR1A | Fc fragment of IgG, high | Fc receptor | Immune | Hs.77424 |
| | | affinity Ia, receptor | | | |
| | | (CD64) | | | |
| CD66b | CEACAM8 | carcinoembryonic antigen- | Cell adhesion | Cell-cell | Hs.41 |
| | | related cell adhesion | | interaction | |
| | | molecule 8 | | | |
| CD66c | CEACAM6 | carcinoembryonic antigen- | Multifunctional | Innate | Hs.466814 |
| | | related cell adhesion | | immunity | |
| | | molecule 6 (non-specific | | | |
| | | cross reacting antigen) | | | |
| CD66f | PSG1 | pregnancy specific beta-1- | Growth | Development, | Hs.709192 |
| | | glycoprotein 1 | factor/hormone | cell growth | |
| CD69 | CD69 | CD69 molecule | Receptor | Immune | Hs.208854 |
| CD70 | CD70 | CD70 molecule | Cytokine/chemokine | Costimulatory | Hs.501497 |
| | | | signaling | | |
| CD80 | CD80 | CD80 molecule | Co-stimulation | Immune | Hs.838 |
| CD86 | CD86 | CD86 molecule | Co-stimulation | Immune | Hs.171182 |
| CD87 | PLAUR | plasminogen activator, | Receptor | Protein | Hs.466871 |
| | | urokinase receptor | | processing | |
| CD93 | CD93 | CD93 molecule | Cell adhesion | Cell-cell | Hs.97199 |
| | | | | interaction | |
| CD98 | SLC7A5 | solute carrier family 7 | Carrier | Transport | Hs.513797 |
| | | (cationic amino acid | | | |
| | | transporter, y+ system), | | | |
| | | member 5 | | | |
| CENPA | CENPA | centromere protein A | DNA binding | Cell cycle | Hs.1594 |
| CENPQ | CENPQ | centromere protein Q | DNA binding | Cell cycle | Hs.88663 |
| CENPT | CENPT | centromere protein T | DNA binding | Cell cycle | Hs.288382 |
| CFB | CFB | complement factor B | Complement | Innate | Hs.69771 |
| | | | | immunity | |
| CHRNA1 | CHRNA1 | cholinergic receptor, | Receptor | Signaling | Hs.434479 |
| | | nicotinic, alpha 1 (muscle) | | | |
| COL1A2 | COL1A2 | collagen, type I, alpha 2 | ECM | Cell adhesion | Hs.489142 |
| COL20A1 | COL20A1 | collagen, type XX, alpha 1 | ECM | Cell adhesion | Hs.271285 |
| COL4A6 | COL4A6 | collagen, type IV, alpha 6 | ECM | Cell adhesion | Hs.145586 |
| COL9A1 | COL9A1 | collagen, type IX, alpha 1 | ECM | Cell adhesion | Hs.590892 |
| COLQ | COLQ | collagen-like tail subunit | ECM | Neurotransmitter | Hs.146735 |
| | | (single strand of | | synthesis/clearance | |
| | | homotrimer) of | | | |
| | | asymmetric | | | |
| | | acetylcholinesterase | | | |
| DDC | DDC | dopa decarboxylase | Enzyme | Neurotransmitter | Hs.359698 |
| | | (aromatic L-amino acid | | synthesis/ | |
| | | decarboxylase) | | clearance | |
| DLAT | DLAT | dihydrolipoamide S- | Enzyme | Metabolism | Hs.335551 |
| | | acetyltransferase | | | |
| CD16a | FCGR3A | Fc fragment of IgG, low | Fc receptor | Immune | Hs.694258 |
| | | affinity IIIa, receptor | | | |
| | | (CD16a) | | | |
| FGF7 | FGF7 | fibroblast growth factor 7 | Growth | Development, | Hs.567268 |
| | | (keratinocyte growth | factor/hormone | cell growth | |
| | | factor) | | | |
| H1F0 | H1F0 | H1 histone family, | DNA binding | Structural | Hs.715673 |
| | | member 0 | | | |
| H2AFX | H2AFX | H2A histone family, | DNA binding | Structural | Hs.477879 |
| | | member X | | | |
| H2AFY | H2AFY | H2A histone family, | DNA binding | Structural | Hs.599225 |
| | | member Y | | | |
| H3F3B | H3F3B | H3 histone, family 3B | DNA binding | Structural | Hs.180877 |
| | | (H3.3B) | | | |
| HBA1 | HBA1 | hemoglobin, alpha 1 | Oxygen binding | Metabolism | Hs.449630 |
| HBA2 | HBA2 | hemoglobin, alpha 2 | Oxygen binding | Metabolism | Hs.449630 |
| HBD | HBD | hemoglobin, delta | Oxygen binding | Metabolism | Hs.699280 |
| HBM | HBM | hemoglobin, mu | Oxygen binding | Metabolism | Hs.647389 |
| HLA-C | HLA-C | major histocompatibility | MHC | Immune | Hs.654404 |
| | | complex, class I, C | | | |
| HLA-DQB1 | HLA-DQB1 | major histocompatibility | MHC | Immune | Hs.409934 |
| | | complex, class II, DQ beta 1 | | | |
| HLA-F | HLA-F | major histocompatibility | MHC | Immune | Hs.519972 |
| | | complex, class I, F | | | |
| HSP90B1 | HSP90B1 | heat shock protein 90 kDa | Chaperone | Protein | Hs.192374 |
| | | beta (Grp94), member 1 | | processing | |
| HSPB7 | HSPB7 | heat shock 27 kDa protein | Chaperone | Protein | Hs.502612 |
| | | family, member 7 | | processing | |
| | | (cardiovascular) | | | |
| IFNG | IFNG | interferon, gamma | Cytokine/chemokine | Immune | Hs.856 |
| | | | signaling | | |
| IGFL2 | IGFL2 | IGF-like family member 2 | Growth | Development, | Hs.99376 |
| | | | factor/hormone | cell growth | |
| IGH2 | IGHE | immunoglobulin heavy | Immunoglobulin | Immune | Hs.700112 |
| | | locus /// ig heavy chain V- | | | |
| | | III region VH26-like | | | |
| IGHA1 /// | IGHG2 or | immunoglobulin heavy | Immunoglobulin | Immune | Hs.460661 |
| IGHD /// | IGHG4 | constant mu /// | | | |
| IGHG1 /// | | hypothetical protein | | | |
| IGHG2 /// | | LOC100132941 /// ig | | | |
| IGHG3 /// | | heavy chain V-III region | | | |
| IGHM /// | | VH26-like | | | |
| IGHV4-31 | | | | | |
| /// | | | | | |
| LOC100132941 | | | | | |
| /// | | | | | |
| LOC100289290 | | | | | |
| /// | | | | | |
| LOC100290036 | | | | | |
| /// | | | | | |
| LOC652494 | | | | | |
| IGHM | IGHM | immunoglobulin heavy | Immunoglobulin | Immune | — |
| | | constant mu | | | |
| IGHV4-31 | Variable Ig | | | | |
| | region | | | | |
| IGHV7-81 | Variable Ig | | | | |
| | region | | | | |
| IL12A | IL12A | interleukin 12A (natural | Cytokine/chemokine | Immune | Hs.673 |
| | | killer cell stimulatory | signaling | | |
| | | factor 1, cytotoxic | | | |
| | | lymphocyte maturation | | | |
| | | factor 1, p35) | | | |
| IL1RAPL2 | IL1RAPL2 | interleukin 1 receptor | Signaling | Immune | Hs.675519 |
| | | accessory protein-like 2 | | | |
| IL6 | IL6 | interleukin 6 (interferon, | Cytokine/chemokine | Immune | Hs.654458 |
| | | beta 2) | signaling | | |
| IL6R | IL6R | interleukin 6 receptor | Cytokine/chemokine | Immune | Hs.709210 |
| | | | signaling | | |
| IL8 | IL8 | interleukin 8 | Cytokine/chemokine | Immune | Hs.624 |
| | | | signaling | | |
| IRF8 | IRF8 | interferon regulatory | Transcription | Gene | Hs.137427 |
| | | factor 8 | factor | expression | |
| ITGA2B | ITGA2B | integrin, alpha 2b (platelet | Cell adhesion | Cell-cell | Hs.411312 |
| | | glycoprotein IIb of IIb/IIIa | | interaction | |
| | | complex, antigen CD41) | | | |
| ITGB3BP | ITGB3BP | integrin beta 3 binding | Cell adhesion | Cell-cell | Hs.166539 |
| | | protein (beta3-endonexin) | | interaction | |
| KRT73 | KRT73 | keratin 73 | Keratin | Structural | Hs.55410 |
| KRT19 | KRT19 | keratin 19 | Keratin | Structural | Hs.654568 |
| KRTAP13-1 | KRTAP13-1 | keratin associated protein | Keratin | Structural | Hs.407653 |
| | | 13-1 | | | |
| KRTAP9-3 | KRTAP9-3 | keratin associated protein | Keratin | Structural | Hs.307012 |
| | | 9-3 | | | |
| KRTAP9-8 | KRTAP9-8 | keratin associated protein | Keratin | Structural | Hs.307011 |
| | | 9-8 | | | |
| MBP | MBP | myelin basic protein | Cell adhesion | Myelination | Hs.551713 |
| MLF1IP | MLF1IP | MLF1 interacting protein | DNA binding | Cell cycle | Hs.575032 |
| MOBP | MOBP | myelin-associated | Cytoskeleton | Trafficking | Hs.121333 |
| | | oligodendrocyte basic | | | |
| | | protein | | | |
| MS4A8B | MS4A8B | membrane-spanning 4- | Poorly | Unknown | Hs.150878 |
| | | domains, subfamily A, | characterized | | |
| | | member 8B | | | |
| MYH9 | MYH9 | myosin, heavy chain 9, | Molecular motor | Contractility | Hs.474751 |
| | | non-muscle | | | |
| MYO1A | MYO1A | myosin IA | Molecular motor | Contractility | Hs.5394 |
| MYO1D | MYO1D | myosin ID | Molecular motor | Contractility | Hs.658000 |
| MYO7B | MYO7B | myosin VIIB | Molecular motor | Contractility | Hs.154578 |
| MYT1L | MYT1L | myelin transcription factor | Transcription | Neuronal | Hs.434418 |
| | | 1-like | factor | development/ | |
| | | | | differentiation | |
| NMNAT2 | NMNAT2 | nicotinamide nucleotide | Enzyme | Metabolism | Hs.497123 |
| | | adenylyltransferase 2 | | | |
| NOL1 | NSUN5 | NOL1/NOP2/Sun domain | Enzyme | DNA | Hs.647060 |
| | | family, member 5 | | methylation | |
| NOLA3 | NOP10 | NOP10 ribonucleoprotein | RNA binding | RNA | Hs.14317 |
| | | homolog (yeast) | | processing | |
| PADI4 | PADI4 | peptidyl arginine | Enzyme | Metabolism | Hs.522969 |
| | | deiminase, type IV | | | |
| PDCD1 | PDCD1 | programmed cell death 1 | Receptor | Immune | Hs.158297 |
| PIP4K2C | PIP4K2C | phosphatidylinositol-5- | Kinase/phosphatase | Signaling | Hs.144502 |
| | | phosphate 4-kinase, type | | | |
| | | II, gamma | | | |
| POLR2C | POLR2C | polymerase (RNA) II | Enzyme | Gene | Hs.79402 |
| | | (DNA directed) | | expression | |
| | | polypeptide C, 33 kDa | | | |
| POLR2H | POLR2H | polymerase (RNA) II | Enzyme | Gene | Hs.432574 |
| | | (DNA directed) | | expression | |
| | | polypeptide H | | | |
| POLR2I | POLR2I | polymerase (RNA) II | Enzyme | Gene | Hs.47062 |
| | | (DNA directed) | | expression | |
| | | polypeptide I, 14.5 kDa | | | |
| POLR2J2 | POLR2J2 | polymerase (RNA) II | Enzyme | Gene | Hs.696339 |
| | | (DNA directed) | | expression | |
| | | polypeptide J2 | | | |
| POLR3D | POLR3D | polymerase (RNA) III | Enzyme | Gene | Hs.148342 |
| | | (DNA directed) | | expression | |
| | | polypeptide D, 44 kDa | | | |
| POLR3H | POLR3H | polymerase (RNA) III | Enzyme | Gene | Hs.712617 |
| | | (DNA directed) | | expression | |
| | | polypeptide H (22.9 kD) | | | |
| PSIP1 | PSIP1 | PC4 and SFRS1 | Transcription | Gene | Hs.658434 |
| | | interacting protein 1 | factor | expression | |
| SRP19 | SRP19 | signal recognition particle | RNA binding | Gene | Hs.637001 |
| | | 19 kDa | | expression | |
| STAT4 | STAT4 | signal transducer and | Signaling | Immune | Hs.80642 |
| | | activator of transcription 4 | | | |
| STK19 | STK19 | serine/threonine kinase 19 | Kinase | Signaling | Hs.654371 |
| TPO | TPO | thyroid peroxidase | Enzyme | Metabolism | Hs.467554 |
| UEVLD | UEVLD | UEV and lactate/malate | Enzyme | Protein | Hs.407991 |
| | | dehyrogenase domains | | turnover | |
| BRCA1 | BRCA1 | breast cancer 1, early | Protein binding | DNA | Hs.194143 |
| | | onset | | replication/ | |
| | | | | repair | |
| CD88 | C5AR1 | complement component | Complement | Innate | Hs.2161 |
| | | 5a receptor 1 | | immunity | |
| CSF2RA | CSF2RA | colony stimulating factor | Receptor | Immune | Hs.520937 |
| | | 2 receptor, alpha, low- | | | |
| | | affinity (granulocyte- | | | |
| | | macrophage) | | | |
| HBZ | HBZ | hemoglobin, zeta | Oxygen binding | Metabolism | Hs.585357 |
| HSPD1 | HSPD1 | heat shock 60 kDa protein | Chaperone | Immune, | Hs.595053 |
| | | 1 (chaperonin) | | innate | |
| | | | | immunity | |
| IFNA7 | IFNA7 | interferon, alpha 7 | Cytokine/chemokine | Immune | Hs.282274 |
| | | | signaling | | |
| IL17D | IL17D | interleukin 17D | Cytokine/chemokine | Immune | Hs.655142 |
| | | | signaling | | |
| KRT17 | KRT17 | keratin 17 | Keratin | Structural | Hs.2785 |
| KRT18 | KRT18 | keratin 18 | Keratin | Structural | Hs.406013 |
| KRT24 | KRT24 | keratin 24 | Keratin | Structural | Hs.87383 |
| KRT5 | KRT5 | keratin 5 | Keratin | Structural | Hs.433845 |
| MYL6 | MYL6 | myosin, light chain 6, | Molecular motor | Contractility | Hs.632717 |
| | | alkali, smooth muscle and | | | |
| | | non-muscle | | | |
| MYO9B | MYO9B | myosin IXB | Molecular motor | Contractility | Hs.123198 |
| PARP12 | PARP12 | poly (ADP-ribose) | Enzyme | DNA | Hs.12646 |
| | | polymerase family, | | replication/repair | |
| | | member 12 | | | |
| CD31 | PECAM1 | platelet/endothelial cell | Cell adhesion | Cell-cell | Hs.514412 |
| | | adhesion molecule | | interaction | |
| POLR3GL | POLR3GL | polymerase (RNA) III | Enzyme | Gene | Hs.591456 |
| | | (DNA directed) | | expression | |
| | | polypeptide G (32 kD)-like | | | |
| SC65 | SC65 | synaptonemal complex | Poorly | Unknown | Hs.446459 |
| | | protein SC65 | characterized | | |
| SLC5A5 | SLC5A5 | solute carrier family 5 | Carrier | Transport | Hs.584804 |
| | | (sodium iodide | | | |
| | | symporter), member 5 | | | |
| UTP14a | UTP14a | UTP14, U3 small | RNA binding | RNA | Hs.458598 |
| | | nucleolar | | processing | |
| | | ribonucleoprotein, | | | |
| | | homolog A (yeast) | | | |
| PTBP1 | PTBP1 | polypyrimidine tract | RNA binding | RNA | Hs.172550 |
| | | binding protein 1 | | processing | |

At least two of the selected antigens preferably have quantified and known relative autoantibody reactivities with respect to sera of a population affected by a disease, as well as a known association with a disease parameter.

In some contemplated embodiments, the carrier can be a pharmaceutically-acceptable carrier, and the composition can be formulated as a vaccine. In such embodiments, it is generally preferred that the vaccine comprises multiple (e.g., at least two, four, or six) antigens. Depending on the particular disease or disorder, it is contemplated that the antigens or fragments thereof can be at least partially purified and/or recombinant.

Alternatively, the carrier could be a solid carrier, and the plurality of antigens could be disposed on the carrier either as a mixture or as an array. In such arrays, it is contemplated that the antigens could have at least two distinct known reactivities and/or parameters. It is contemplated that the antigens or fragments thereof can be in crude expression extracts, in partially purified form (e.g., purity of less than 60%), or in highly purified form (e.g., purity of at least 95%). The antigens in such arrays may be recombinant or native. Alternatively, the solid phase need not be limited to planar arrays, but could also include, for example, beads, columns, dipstick-type formats, and other commercially suitable media.

In an alternative embodiment, two or more of the antigens can be immobilized on a surface, and the antigens can be associated with a single disease or more than one disease.

The surface can alternatively have antigen variants including, for example, truncated forms, non-glycosylated forms, recombinant forms, and chimeric forms.

In some contemplated embodiments, the disease is breast cancer, and the plurality of antigens are selected from the group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, and UTP14a, or fragments thereof.

In other contemplated embodiments, the disease is lupus (L), and wherein the plurality of antigens are selected from the group consisting of DPP4, IL6R, ITGB2, MLF1IP, MYO1A, POLR2H, and TPO, or fragments thereof. In still other contemplated embodiments, the disease is lupus nepritis (LN), and wherein the plurality of antigens are selected from the group consisting of CD1D, IL6R, IRF8, ITGA2B, MYO1A, MYO7B, PSG1, PTBP1, and TPO, or fragments thereof.

In yet another contemplated embodiment, the disease can be systemic lupus erythematosus (SLE), and wherein the plurality of antigens are selected from the group consisting of CD1C, CD46, CENPQ, CFB, DPP4, HLA-DQB1, IL6R, ITGB2, KRTAP9-3, MLF1IP, MYT1L, POLR2H, SLC7A5, and TPO, or fragments thereof. In still another contemplated embodiment, the disease can be scleroderma (Sc) and the antigen can be IL6R, or a fragment thereof.

In other contemplated embodiments, the disease can be polymyositis (P), and wherein the plurality of antigens are selected from the group consisting of CD14, CD1C, CD46, CD55, CFB, COL9A1, COLQ, DLAT, DPP4, FGF7, H3F3B, IL1RAPL2, IL6R, IL8, ITGB2, KRTAP9-3, MLF1IP, MYT1L, PADI4, PIP4K2C, PLAUR, POLR2H, POLR2I, PSG1, SLC7A5, and STK19, or fragments thereof.

In an alternative embodiment, the disease can be rheumatoid arthritis (RA), and wherein the plurality of antigens are selected from the group consisting of APOH, BANK1, BLK, CD1C, CD14, CD3E, CD70, CD80, CD86, CEACAM6, CEACAM8, CENPT, CFB, COL1A2, DDC, DPP4, FCGR1A, H2AFX, H2AFY, H3F3B, HBA1, HBA2, HBD, HLA-DQB1, HSP90B1, HSPB7, IGHG2, IGHG4, IGHM, IGHV4-31, IL12A, IL6, IL6R, ITGB3BP, KRTAP13-1, KRTAP9-3, MBP, MLF1IP, MOBP, MS4A8B, MYH9, MYO1D, MYT1L, NMNAT2, NOL1, PDCD1, PIP4K2C, POLR2C, POLR2H, POLR2I, POLR2J2, POLR3D, PSIP1, SRP19, STAT4, and STK19, or fragments thereof.

In another embodiment, the disease can be Sjögren's syndrome (Sj), and wherein the plurality of antigens are selected from the group consisting of APOH, CALR3, CD1C, CD14, CD34, CD3E, CD46, CD69, CD93, CEACAM8, CENPA, CENPQ, CFB, CHRNA1, COL20A1, COL4A6, DPP4, FCGR3A, H1F0, H2AFX, H3F3B, HBA1, HBA2, HBD, HBM, HLA-C, HLA-DQB1, HLA-F, HSPB7, IFNG, IGFL2, IGH2, IGHV7-81, IL1RAPL2, IL6R, ITGB2, keratin 73, KRT19, KRTAP9-3, KRTAP9-8, MBP, MLF1IP, MYT1L, NOLA3, POLR2H, POLR2I, POLR3D, POLR3H, PTBP1, STK19, and UEVLD, or fragments thereof.

A list of diseases and antigen groups can be found in Table 2 below.

TABLE 2

| L | SLE | LN | P | RA | Sc | Sj |
|---|---|---|---|---|---|---|
| CFB | CD1C | CD1D | CD14 | APOH | IL6R | APOH |
| DPP4 | CD46 | IL6R | CD1C | BANK1 | | CALR3 |
| IL6R | CENPQ | IRF8 | CD46 | BLK | | CD1C |
| ITGB2 | CFB | ITGA2B | CD55 | CD1C | | CD14 |
| MLF1IP | DPP4 | MYO1A | CFB | CD14 | | CD34 |
| MYO1A | HLA-DQB1 | MYO7B | COL9A1 | CD3E | | CD3E |
| POLR2H | IL6R | PSG1 | COLQ | CD70 | | CD46 |
| TPO | ITGB2 | PTBP1 | DLAT | CD80 | | CD69 |
| | KRTAP9-3 | TPO | DPP4 | CD86 | | CD93 |
| | MLF1IP | | FGF7 | CEACAM6 | | CEACAM8 |
| | MYT1L | | H3F3B | CEACAM8 | | CENPA |
| | POLR2H | | IL1RAPL2 | CENPT | | CENPQ |
| | SLC7A5 | | IL6R | CFB | | CFB |
| | TPO | | IL8 | COL1A2 | | CHRNA1 |
| | | | ITGB2 | DDC | | COL20A1 |
| | | | KRTAP9-3 | DPP4 | | COL4A6 |
| | | | MLF1IP | FCGR1A | | DPP4 |
| | | | MYT1L | H2AFX | | FCGR3A |
| | | | PADI4 | H2AFY | | H1F0 |
| | | | PIP4K2C | H3F3B | | H2AFX |
| | | | PLAUR | HBA1 | | H3F3B |
| | | | POLR2H | HBA2 | | HBA1 |
| | | | POLR2I | HBD | | HBA2 |
| | | | PSG1 | HLA-DQB1 | | HBD |
| | | | SLC7A5 | HSP90B1 | | HBM |
| | | | STK19 | HSPB7 | | HLA-C |
| | | | | IGHG2 | | HLA-DQB1 |
| | | | | IGHG4 | | HLA-F |
| | | | | IGHM | | HSPB7 |
| | | | | IGHV4-31 | | IFNG |
| | | | | IL12A | | IGFL2 |
| | | | | IL6 | | IGH2 |
| | | | | IL6R | | IGHV7-81 |
| | | | | ITGB3BP | | IL1RAPL2 |
| | | | | KRTAP13-1 | | IL6R |
| | | | | KRTAP9-3 | | ITGB2 |
| | | | | MBP | | keratin 73 |
| | | | | MLF1IP | | KRT19 |
| | | | | MOBP | | KRTAP9-3 |
| | | | | MS4A8B | | KRTAP9-8 |
| | | | | MYH9 | | MBP |
| | | | | MYO1D | | MLF1IP |
| | | | | MYT1L | | MYT1L |
| | | | | NMNAT2 | | NOLA3 |
| | | | | NOL1 | | POLR2H |
| | | | | PDCD1 | | POLR2I |
| | | | | PIP4K2C | | POLR3D |
| | | | | POLR2C | | POLR3H |
| | | | | POLR2H | | PTBP1 |
| | | | | POLR2I | | STK19 |
| | | | | POLR2J2 | | UEVLD |
| | | | | POLR3D | | |
| | | | | PSIP1 | | |
| | | | | SRP19 | | |
| | | | | STAT4 | | |
| | | | | STK19 | | |

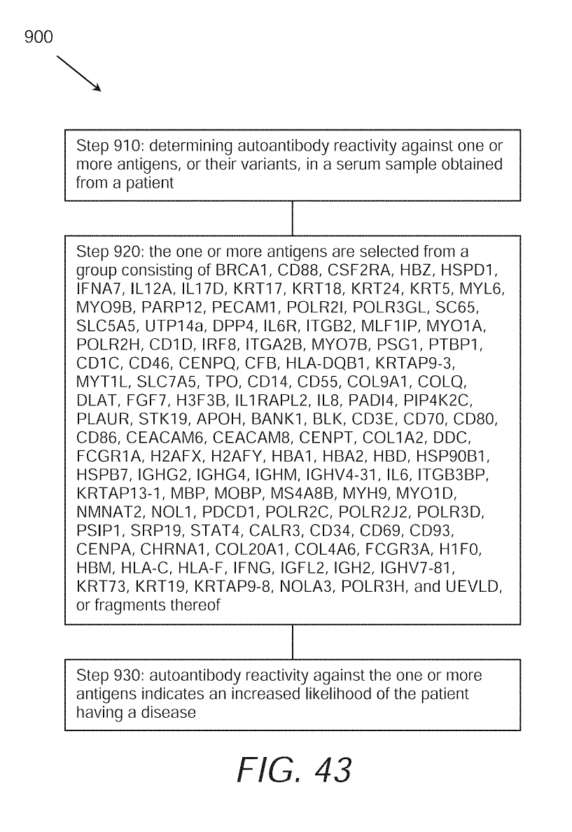

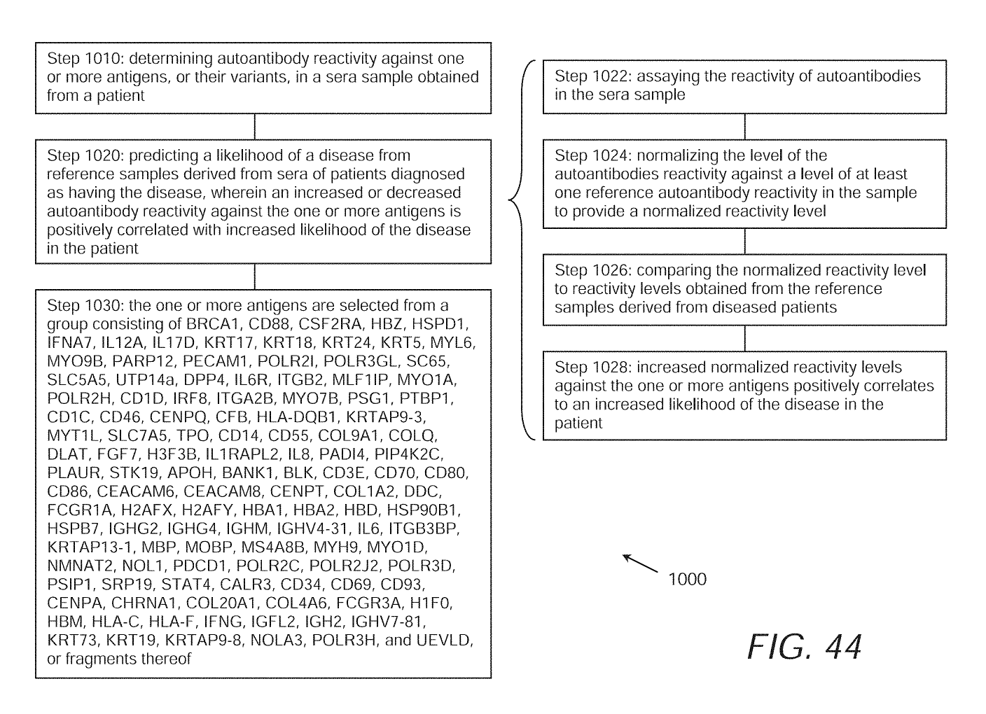

In FIG. 43, one embodiment of a method 900 for predicting the likelihood of a patient having a disease or disorder can include step 910 of determining autoantibody reactivity against one or more antigens, or their variants, in a serum sample obtained from a patient. In step 920, the one or more antigens are preferably selected from the group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, UTP14a, DPP4, IL6R, ITGB2, MLF1IP, MYO1A, POLR2H, CD1D, IRF8, ITGA2B, MYO7B, PSG1, PTBP1, CD1C, CD46, CENPQ, CFB, HLA-DQB1, KRTAP9-3, MYT1L, SLC7A5, TPO, CD14, CD55, COL9A1, COLQ, DLAT, FGF7, H3F3B, IL1RAPL2, IL8, PADI4, PIP4K2C, PLAUR, STK19, APOH, BANK1, BLK, CD3E, CD70, CD80, CD86, CEACAM6, CEACAM8, CENPT, COL1A2, DDC, FCGR1A, H2AFX, H2AFY, HBA1, HBA2, HBD, HSP90B1, HSPB7, IGHG2, IGHG4, IGHM, IGHV4-31, IL6, ITGB3BP, KRTAP13-1, MBP, MOBP, MS4A8B, MYH9, MYO1D, NMNAT2, NOL1, PDCD1, POLR2C, POLR2J2, POLR3D, PSIP1, SRP19, STAT4, CALR3, CD34, CD69, CD93, CENPA, CHRNAL COL20A1, COL4A6, FCGR3A, H1F0, HBM, HLA-C, HLA-F, IFNG, IGFL2, IGH2, IGHV7-81, KRT73, KRT19, KRTAP9-8, NOLA3, POLR3H, and UEVLD, or fragments thereof.

Determining the autoantibody reactivity against the selected antigens or their variants in step 930 can advantageously indicate an increased likelihood of the patient having a disease, and can thereby provide a manner to detect one or more diseases in a patient. Depending upon the specific disease(s) to be identified, different antigens can be selected.

For example, to predict the likelihood of a patient having breast cancer, the step of determining autoantibody reactivity against one or more antigens or their variants can utilize one or more antigens selected from the group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, and UTP14a, or fragments thereof. In this manner, antibody reactivity against one or more of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, and UTP14a, or fragments thereof, can indicate an increased likelihood of the patient having breast cancer.

As another example, to identify patients with lupus or the likelihood of a patient to have lupus, the one or more antigens are selected from the group consisting of DPP4, IL6R, ITGB2, MLF1IP, MYO1A, POLR2H, and TPO, or fragments thereof. Autoantibody reactivity can then be determined against the selected antigens or their variants, which can advantageously indicate an increased likelihood of the patient having lupus.

To identify patients with lupus nephritis or the likelihood of a patient to have lupus nephritis, the one or more antigens are preferably selected from the group consisting of CD1D, IL6R, IRF8, ITGA2B, MYO1A, MYO7B, PSG1, PTBP1, and TPO, or fragments thereof, and autoantibody reactivity can then be determined against the selected antigens or their variants to thereby indicate the likelihood of the patient having lupus nephritis.

To identify patients with systemic lupus erythematosus or predict the likelihood of a patient having systemic lupus erythematosus, the one or more antigens are preferably selected from the group consisting of CD1C, CD46, CENPQ, CFB, DPP4, HLA-DQB1, IL6R, ITGB2, KRTAP9-3, MLF1IP, MYT1L, POLR2H, SLC7A5, and TPO, or fragments thereof. Autoantibody reactivity can then be determined against the selected antigens or their variants, which can advantageously indicate an increased likelihood of the patient having systemic lupus erythematosus.

As yet another example, to identify patients with polymyositis, it is preferred that the antigens are selected from the group consisting of CD14, CD1C, CD46, CD55, CFB, COL9A1, COLQ, DLAT, DPP4, FGF7, H3F3B, IL1RAPL2, IL6R, IL8, ITGB2, KRTAP9-3, MLF1IP, MYT1L, PADI4, PIP4K2C, PLAUR, POLR2H, POLR2I, PSG1, SLC7A5, and STK19, or fragments thereof. Autoantibody reactivity can then be determined against the selected antigens or their variants, which can advantageously indicate an increased likelihood of the patient having polymyositis.

As a further example, to identify patients with rheumatoid arthritis, it is preferred that the antigens are selected from the group consisting of APOH, BANK1, BLK, CD1C, CD14, CD3E, CD70, CD80, CD86, CEACAM6, CEACAM8, CENPT, CFB, COL1A2, DDC, DPP4, FCGR1A, H2AFX, H2AFY, H3F3B, HBA1, HBA2, HBD, HLA-DQB1, HSP90B1, HSPB7, IGHG2, IGHG4, IGHM, IGHV4-31, IL12A, IL6, IL6R, ITGB3BP, KRTAP13-1, KRTAP9-3, MBP, MLFIIP, MOBP, MS4A8B, MYH9, MYO1D, MYT1L, NMNAT2, NOL1, PDCD1, PIP4K2C, POLR2C, POLR2H, POLR2I, POLR2J2, POLR3D, PSIP1, SRP19, STAT4, and STK19, or fragments thereof. Autoantibody reactivity can then be determined against the selected antigens or their variants, which can advantageously indicate an increased likelihood of the patient having rheumatoid arthritis.

To identify patients with scleroderma, it is preferred that the selected antigen is IL6R, or a fragment thereof. Autoantibody reactivity can then be determined against IL6R or its variants, which can advantageously indicate an increased likelihood of the patient having scleroderma.

To identify patients with Sjögren's syndrome, it is preferred that the antigens are selected from the group consisting of APOH, CALR3, CD1C, CD14, CD34, CD3E, CD46, CD69, CD93, CEACAM8, CENPA, CENPQ, CFB, CHRNA1, COL20A1, COL4A6, DPP4, FCGR3A, H1F0, H2AFX, H3F3B, HBA1, HBA2, HBD, HBM, HLA-C, HLA-DQB1, HLA-F, HSPB7, IFNG, IGFL2, IGH2, IGHV7-81, IL1RAPL2, IL6R, ITGB2, keratin 73, KRT19, KRTAP9-3, KRTAP9-8, MBP, MLF1IP, MYT1L, NOLA3, POLR2H, POLR2I, POLR3D, POLR3H, PTBP1, STK19, and UEVLD, or fragments thereof. Autoantibody reactivity can then be determined against the selected antigens or their variants, which can advantageously indicate an increased likelihood of the patient having Sjögren's syndrome.

In various embodiments, the reactivity level of at least 2, or at least 5, or at least 10, or at least 15, or at least 20, or at least 25 autoantibodies can be determined. Determining reactivity can be performed in numerous formats that are well known in the art. However, it is generally preferred that the determination is accomplished in a multiplex format, and especially in an array or “strip” format including, for example, arrays, or “strips” having at least one, more typically at least two, and even more typically at least 5, or at least 10, or at least 15, or at least 20, or at least 25 antigens.

FIG. 44 illustrates a flowchart of another embodiment of a method 1000 of detecting a disease in a patient includes step 1010 of determining autoantibody reactivity against one or more antigens, or their variants, in a sera sample obtained from a patient.

A likelihood of a disease can be predicted in step 1020 from reference samples derived from sera of patients diagnosed as having the disease, such that increased or decreased autoantibody reactivity against antigens selected from the group discussed above can be positively correlated in step 1030 with an increased likelihood of a disease in the patient.

The method can further include step 1022 of assaying the reactivity of autoantibodies in the sample, and step 1024 of normalizing the level of the reactivity against a level of at least one reference autoantibody reactivity in the sample to provide a normalized reactivity level.

The normalized reactivity level can then be compared in step 1026 with reactivity levels obtained from the reference samples derived from diseased patients. In this manner, increased normalized reactivity levels against antigens selected from the group of antigens listed in Table 1 positively correlates to an increased likelihood of a disease in the patients in step 1028.

In other contemplated embodiments, a method of predicting the likelihood of a patient having a disease or disorder can include determining prognostic autoantibody reactivity against one or more specific antigens, or their variants, such as those described in Table 1, in a serum sample obtained from the patient, which can be normalized against the level of non-prognostic autoantibody reactivity in the serum sample, or of a reference set of autoantibody reactivity. Autoantibody reactivity against one or more of said specific antigens can be used to indicate an increased likelihood of the patient having a disease or disorder.

In an alternative embodiment, a method of predicting the likelihood of a patient having cancer can include determining the reactivity levels of autoantibodies against antigens, or their variants, presented hereinabove in a serum sample obtained from the patient, which is optionally normalized against the reactivity levels of other autoantibodies against antigens, or their variants, in said sera sample, or of a reference set of autoantibody reactivity levels. The data obtained in step (a) can be subjected to statistical analysis, and a likelihood of the patient having cancer can thus be determined.

In another embodiment, methods of preparing a personalized proteomics and autoantibody profile for a patient are contemplated, which include subjecting a sera sample from the patient to protein array chip analysis. The reactivity level of one or more autoantibodies can be determined against antigens or their variants (e.g., those listed in Table 1), and the reactivity level can optionally be normalized against control reactivity levels. A report can be created summarizing the data obtained by the analysis. Optionally, the report may include a prediction of the likelihood of severity of cancer in the patient and/or a recommendation for a treatment modality of the patient.

In a further aspect, methods for detecting one or more endogenous antibodies in a patient. In a still further aspect, methods are contemplated for detecting one or more autoantibodies in a patient.

In another aspect, antigens that triggered autoantibody reactivities are included in an antigen composition having two or more reactive antigens of a human disease or disorder and are associated with a carrier. The antigens can have quantified and known relative reactivities with respect to sera of a population infected with the organism, and can also have a known association with a disease parameter. Most preferably, the antigens are polypeptides or fragments thereof.

### EXAMPLE 1

Human protein antigens in the following categories were selected for printing on the microarrays: (i) established autoantigens from autoimmune rheumatic diseases; (ii) established autoantigens from organ-specific autoimmune diseases; (iii) autoimmune disease associated molecules as described in recent literature (e.g. MHC molecules, complement components, signaling molecules); (iv) immunological targets with disease modifying potential (e.g. cytokines, chemokines, associated receptors, co-stimulatory molecules, etc.); and (v) proteins with no known immune reactivity (as controls). In total 797 proteins were selected for these experiments.

Human gene clones were obtained from the National Institutes of Health's (NIH) Mammalian Gene Collection (MGC) as cDNA clones. Amplicons of the human genes were obtained by PCR amplification of human genes from the cDNA clones. The primers (Sigma-Aldrich™ in St. Louis, Mo.) were made up of 20 base pairs (BPs) of gene-specific sequences and 20 BPs of adapter sequences. The adapter sequences were configured to be homologous to the cloning site of the linearized T7 expression vector pXT7 and allowed the PCR products to be cloned by homologous recombination in Escherichia coli DH5a cells. A polyhistidine (poly-His) fragment was incorporated at the 5′ end of the fusion protein. The amplicons with the flanking adapter sequences were used for in vivo recombination cloning into a T7 promoter based plasmid expression vector.

After the expressible library was verified to contain the correct inserts, the plasmids with human open reading frames (ORFs) were expressed using an in vitro transcription-translation system following the manufacturer's instructions (RTS 100 kit by Roche™ of Indianapolis, Ind.). Microarrays were printed onto nitrocellulose coated glass FAST slides (Whatman Inc.™ of Piscataway, N.J.) using an OmniGrid AccentTM microarray printer (DigiLab Inc.™ of Holliston, Mass.). Protein expression levels were monitored in the microarrays using anti-poly-His (clone His-1 by Sigma-Aldrich™ in St. Louis, Mo.) and anti-HA antibodies (clone 3F10 by Roche™ of Indianapolis, Ind.). The microarrays were blocked using 1×-blocking buffer (Whatman™, Sanford, Me.) for 30 minutes while the serum samples were pre-incubating. The blocking buffer was removed and the diluted antibodies were added to the microarrays and hybridized overnight in a humidified box.

The next day, the arrays were washed three times with Tris buffer-0.05% Tween-20, and the slides were incubated with biotin-conjugated goat anti-mouse, or biotin-conjugated goat anti-rat, immunoglobulin diluted 1/1,000 in blocking buffer. Secondary antibodies were added to the slides and incubated for one hour at room temperature. Following washing three times with Tris buffer-Tween 20, bound antibodies were detected by incubation with streptavidin-conjugated Sensilight P3 (Columbia Biosciences™ of Columbia, Md.). Following washing as before, additional three washes with Tris buffer saline, and a rinse with ultrapure water (18.2 Ohm), the slides were air dried under centrifugation and examined using a Perkin Elmer ScanArrray Express HT™ microarray scanner (Waltham, Mass.). Intensities were quantified using QuantArray™ software with measured values at each spot equaling the intensity at each spot minus the local background average.

While the study of human pathogens on microarray and related platforms has been successful, there was a lack of data or guidance in the art to support the use of the platforms detailed herein to study human diseases, cancers, or autoimmune disorders. To test whether autoantibodies would recognize antigens on the instant microarrays, the inventors chose to test tumor associated antigens (TAAs) for which there was current literature available. To create a TAA human protein microarray chip (TAA chip), the inventors chose 34 human proteins that had been shown to be autoantigens associated with cancer. The inventors surveyed breast cancer patients, population controls, and blood sister control sera on the TAA chip.

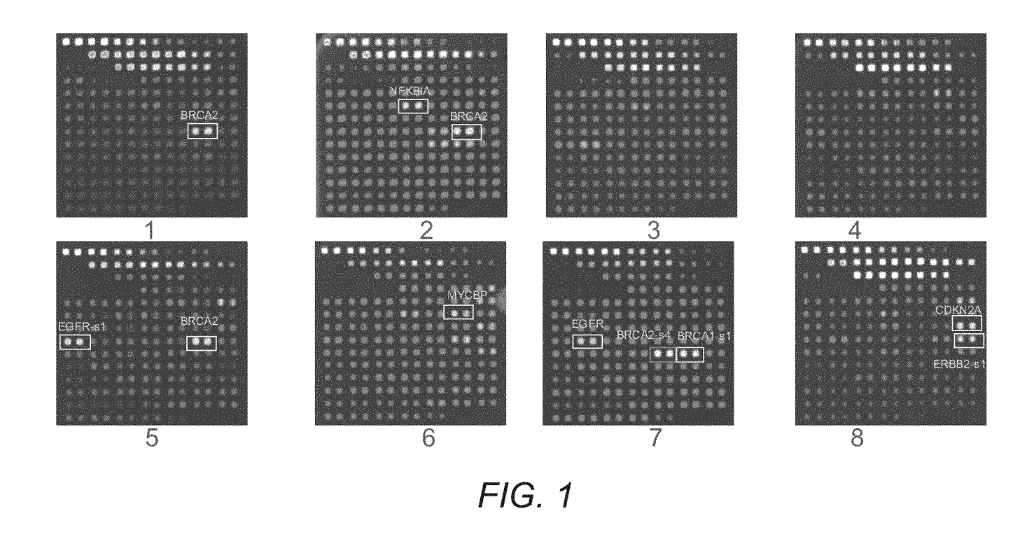

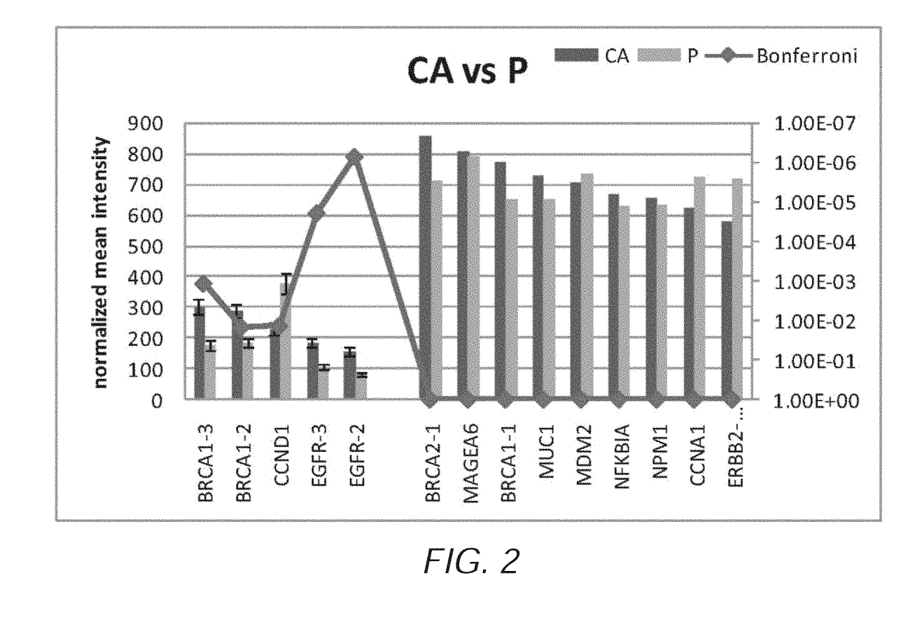

As shown in FIG. 1, the TAA chip was probed with serum from breast cancer patients and controls, and several proteins were recognized by antibodies in the serum (Panels 1-2 and 5-8). Specifically, the antibodies detected included, for example, breast 1 and 2 proteins (BRCA1 and BRCA2) and the epidermal growth factor receptor (EGFR) and EGFR-associated protein erythroblastic leukemia viral oncogene homolog 2 (ERBB2). The image data was quantified and analyzed. FIG. 2 is a histogram comparing the mean signal intensity of the cancer patients (CA) with the population controls (P) and the Bonferroni corrected p-value (Bonferroni). As shown in the histogram, BRCA1 and EGFR were recognized differentially by breast cancer patient sera and the population control sera.

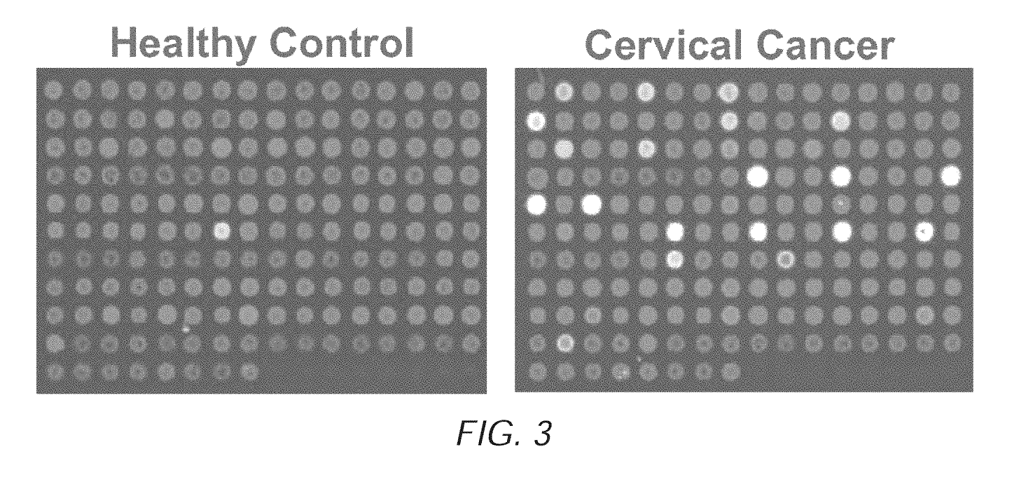

In a parallel study, the TAA chip was probed with serum from patients with cervical cancer and a healthy control. As shown in FIG. 3, there was a stark difference in the profile of antibodies against these TAAs in cervical cancer patients (right panel) compared with a healthy control (left panel). These data suggest that the instant platforms could not only detect autoantibodies, but could be used to determine antibody profiles in cancer patients, patients suffering from autoimmune disorders, and healthy individuals.

Armed with this unexpected success, the inventors created a comprehensive human protein array that would include even more proteins, and then interrogated the microarray with well-characterized, and clinically defined, human serum. Access to a well-characterized set of lupus serum was obtained, and a selection of human proteins to place on the microarray was completed. This Human Autoimmunity Chip (HA or HA1) consisted of 513 human proteins that included 442 unique proteins (the 34 TAAs discussed above and 409 proteins possibly associated with various autoimmune diseases).

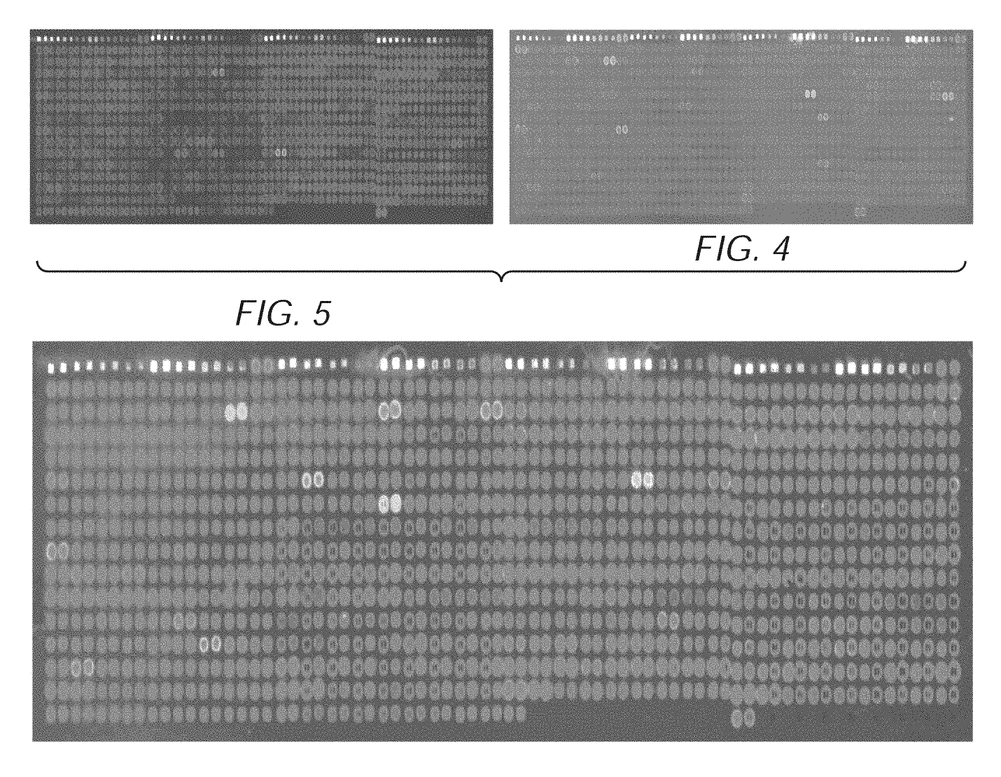

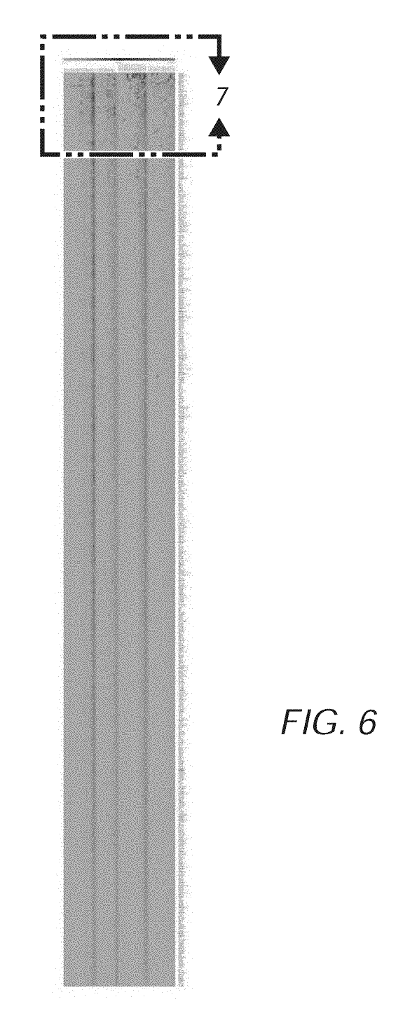

Thirty-one lupus samples were probed including 15 systemic lupus erythematosus (SLE) samples, 16 lupus nephritis (LN) samples, 11 disease control samples (Sjögren's Syndrome (Sj)), and 16 normal controls. FIG. 4 illustrates representative images of the HA chip probed with serum samples from patients with Sj as a disease control, and FIG. 5 illustrates a representative image of the HA chip probed with serum samples from patients with Lupus.

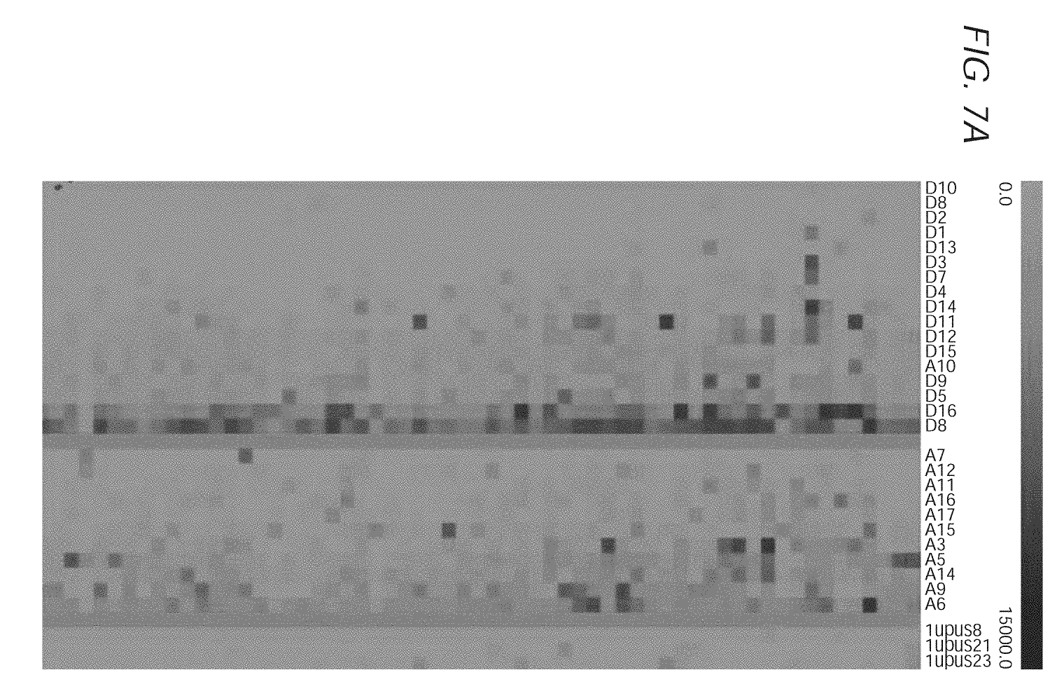

FIG. 6 is a heat map of the signal intensity data for 59 serum samples (columns) and the proteins on the chip (rows) was created, which shows a difference in the reactivity pattern. The most reactive autoantigens in the serum samples from patients with lupus are shown in the enlarged portion of the heat map in FIGS. 7A-7B. Unlike infectious diseases where a naïve (healthy) patient population shows little to no reactivity, there was significant reactivity to human proteins even in the normal/healthy populations. The outcome of the microarray testing showed a difference in the antigen recognition profile of LN and SLE samples when compared to control populations control sera.

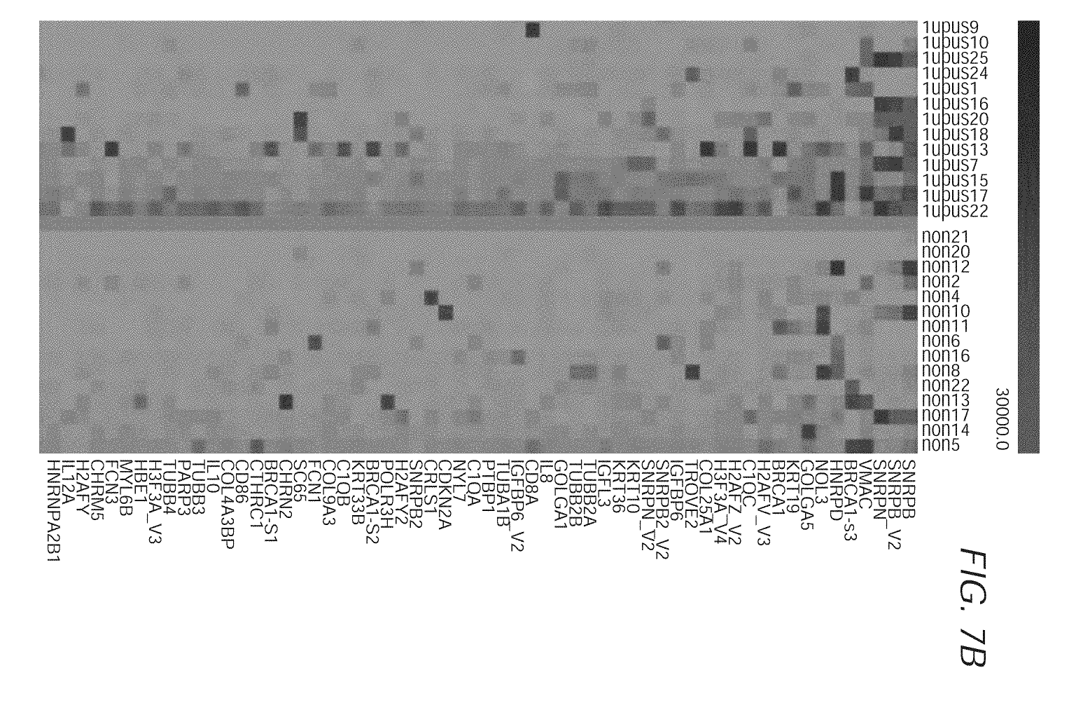

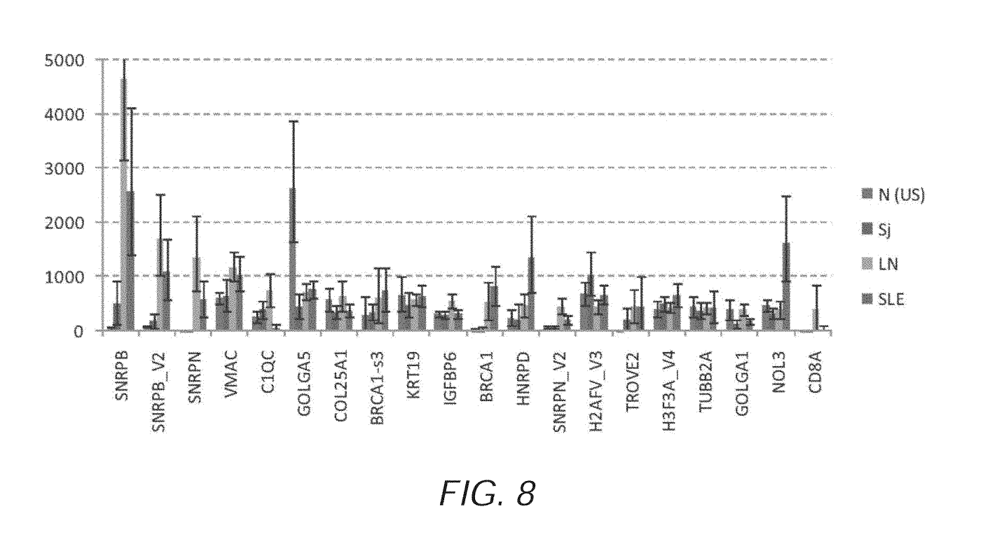

FIG. 8 compares the mean signal intensities and standard errors of normal/healthy sera sourced from the US (N), the Sjögren's Syndrome patient sera (Sj), the lupus nephritis patient sera (LN) and the systemic lupus erythematosus (SLE). Further analysis revealed that there were circulating antibodies against small nuclear ribonucleoprotein polypeptides B, B1 and N (SNRPB and SNRPN), as well as to breast cancer antigen 1 (BRCA1), which are higher in both lupus groups than in the control groups. Having established that the platform could effectively detect circulating antibodies against human proteins, further experiments were conducted to expand the autoimmunity antigen sets and test the discovery platform with a much larger set of characterized samples from lupus patients.

### EXAMPLE 2

### Autoimmune Study

For the second version of the Human Autoimmunity Chip (HA2), an additional 218 proteins were targeted which had 109 splice variants in the MGC, totaling 327 additional proteins. HA2 was composed of 840 total human proteins, representing 660 unique proteins and their splice and/or cDNA variants. To interrogate this expanded set of proteins, serum samples were obtained from patients that had been diagnosed with LN (N=61), SLE (N=72), polymyositis (P) (N=26), rheumatoid arthritis (RA) (N=25), Scleroderma (Sc) (N=21) and Sj (N=23). Serum samples were also obtained from age- and sex-matched normal, healthy individuals (N) (N=10).

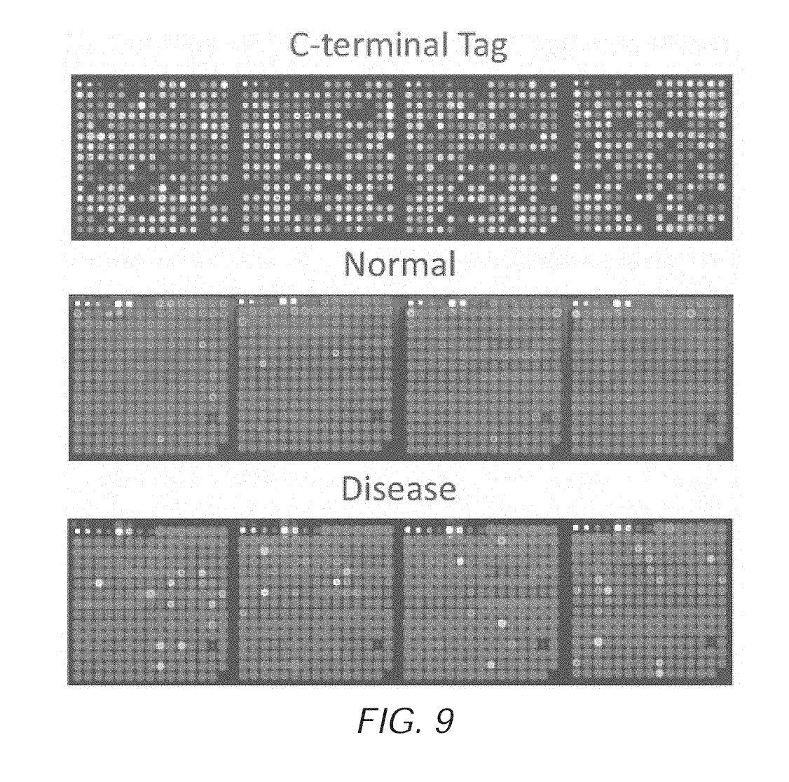

The second version of the HA chip (HA2) was probed with anti-HA high affinity rat monoclonal to verify expression of the proteins. FIG. 9 illustrates sample images of HA2, in which the C-terminal HA tag (top panel) was detected and probed with normal sera (middle panel) and with sera from an autoimmune patient (bottom panel).

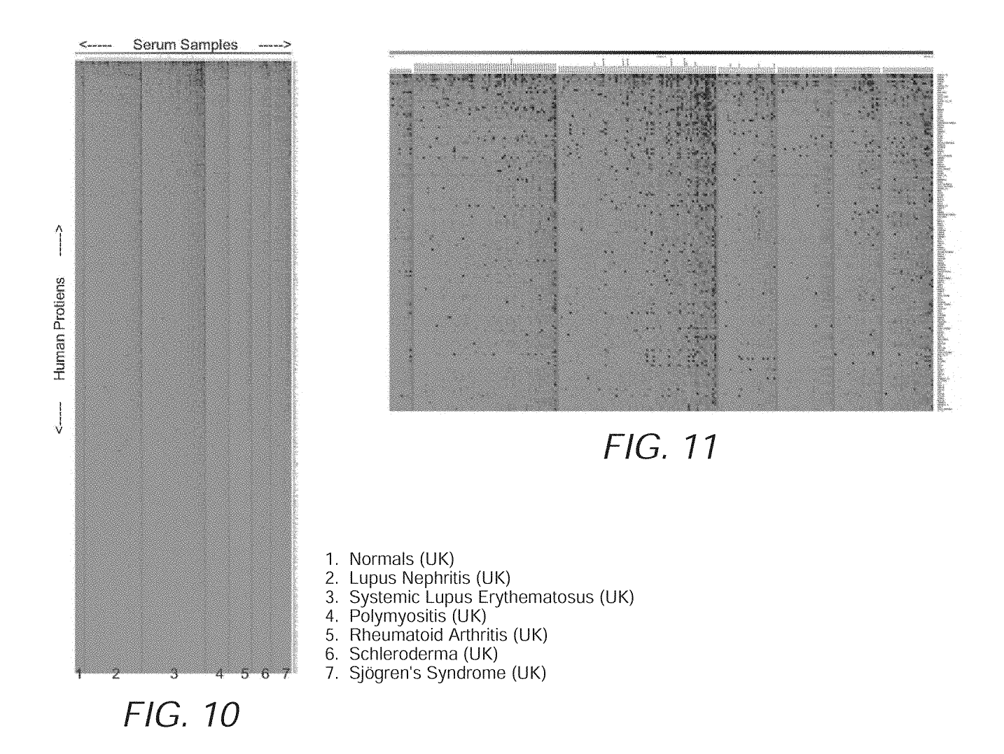

The chips were scanned and quantified using PerkinElmer ProscanArray Express™ v.4 software. The data from the mean-background columns was used to compile the raw data. FIG. 10 is a heat map of the signal intensity data for the approximately 200,000 data points generated from the raw data to examine the data of reactivity patterns of the 238 serum samples (columns) and 840 proteins on the chip (rows). The heat map shows the autoantibody profile for patients with LN (N=61), patients with SLE (N=72), patients with P (N=26), patients with RA (N=25), patients with Sc (N=21), patients with Sj (N=23), and age and sex matched normal individuals (N=10). As better shown in FIG. 11, the heat map illustrates circulating antibodies to human proteins in normal individuals as well as in the disease groups, and shows the antigens that demonstrated the highest signals in SLE.

The data was dissected further to identify disease-specific biomarkers, and the raw data was normalized using variance stabilization normalization (VSN), which is an accepted method to deal with microarray data. Using the normalized data, mean signal intensities and the standard deviation and standard errors were calculated for each group of samples in the statistical environment known as R (www.r-project.org). To determine which antigens were potential biomarkers, the disease groups were compared with the normal group (N) using Benjamini-Hochberg corrected p-values (BHp) calculated from Bayesian regularized t-tests performed in R. To control for multiple testing conditions, p-values were adjusted using the Benjamini-Hochberg procedure for controlling the Family Wise Error Rate. All reported p-values are Benjamini-Hochberg corrected unless otherwise noted. Finally, the data was retransformed into an approximate raw scale by taking the base 2 anti-log of the values for bar plot visualizations.

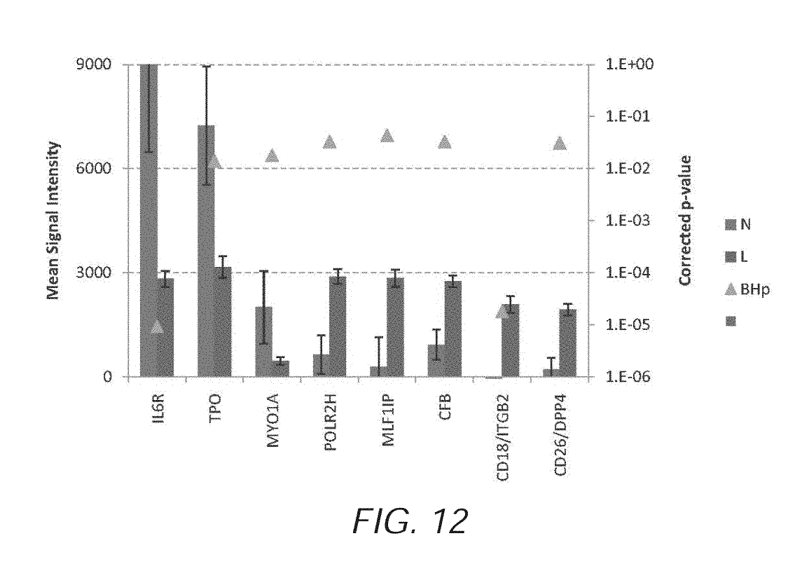

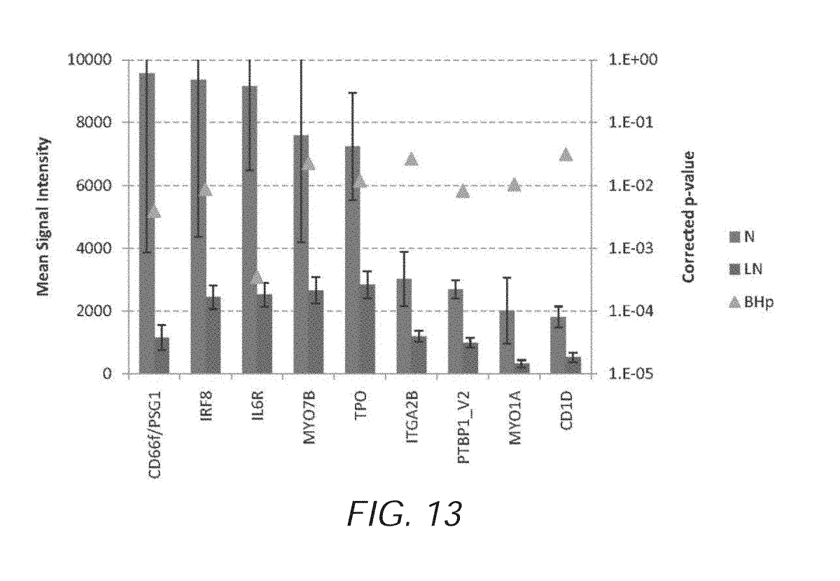

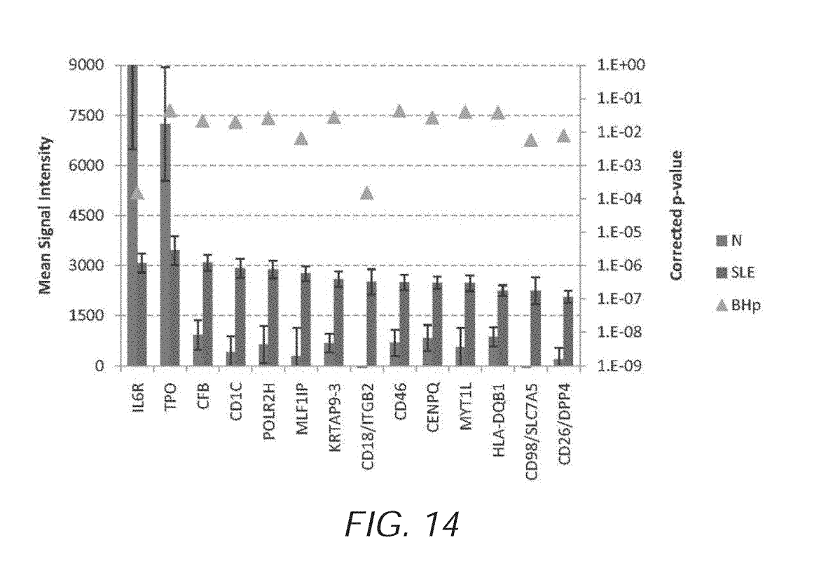

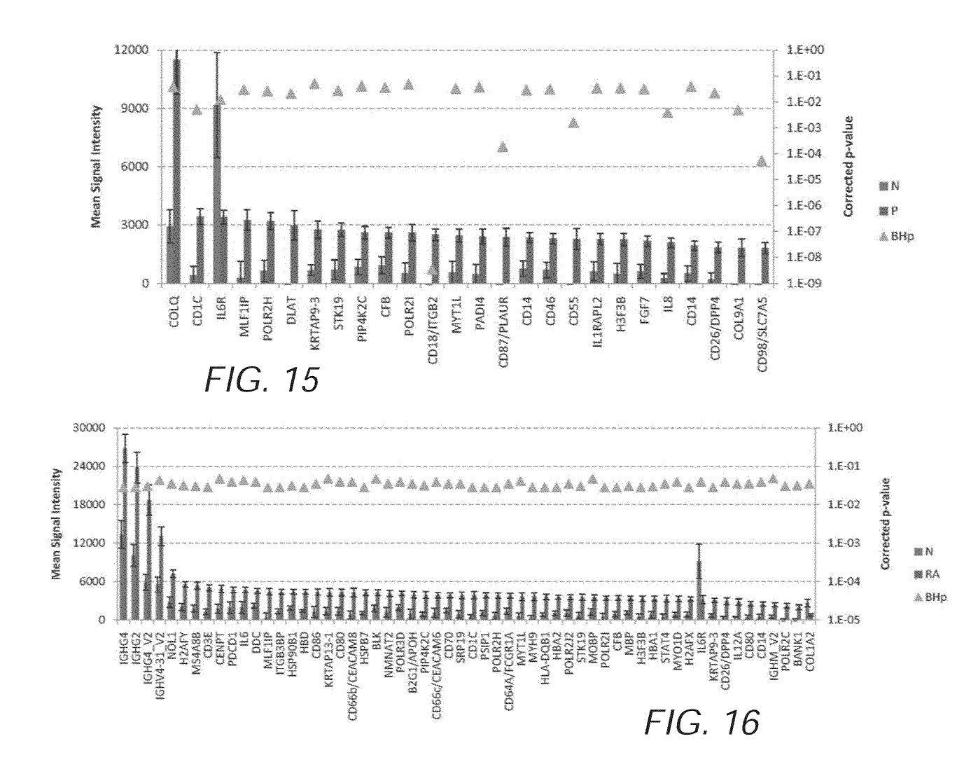

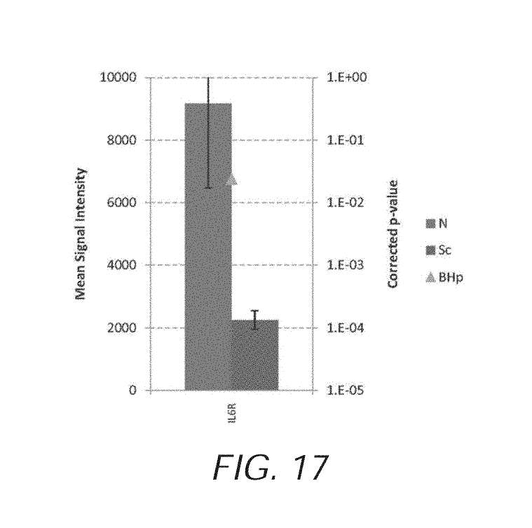

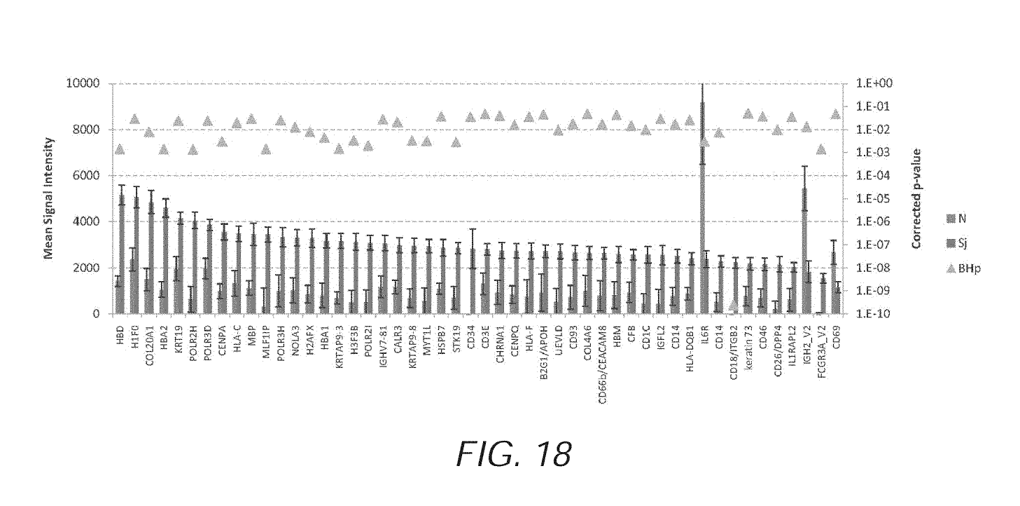

The mean signal intensities and standard errors were plotted for antigens that are differentially reactive when compared to the normal group. Antibody profiles to human proteins associated to specific diseases were readily identified, as shown in FIGS. 12-18. Specifically, FIG. 12 looks at sera from lupus patients (L), FIG. 13 looks at sera from lupus nephritis patients (LN), FIG. 14 looks at sera from systemic lupus erythematosus patients (SLE), FIG. 15 looks at sera from polymyositis patients (P), FIG. 16 looks at sera from rheumatoid arthritis patients (RA), FIG. 17 looks at sera from scleroderma patients (Sc), and FIG. 18 looks at sera from Sjögren's Syndrome patients (Sj).

When reviewing autoantibodies profiles, there are generally two potential outcomes of human disease: either an increase in circulating antibodies or a decrease in existing antibodies. As shown in FIGS. 13 and 17 for LN and Sc, a significant decrease can be seen in circulating antibodies, whereas FIGS. 12, 14-16, and 18 shows a net increase in the circulating antibodies for SLE, P, RA and Sj.

As can be seen in FIGS. 13 and 14, SLE patients have higher reactivity for CFB, (CD1C), POLSR2H, MLF1IP, keratin associated protein 9-3 (KRTAP9-3), ITGB, CD46 molecule (CD46), centromere protein Q (CENPQ), myelin transcription factor 1-like (MYT1L), major histocompatibility complex class II DQ beta 1 (HLA-DQB1), solute carrier family 7 (cationic amino acid transporter, y+ system) member 5 (SLC7A5) and DPP4. IL6R and TPO show lower reactivity in the SLE patients. Seven of the eight proteins show the same pattern of reactivity as seen for all lupus patients. Using the optimal linear combination of nine proteins, a receiver operator curve was created with an area under the curve of 0.990, sensitivity of 98% and specificity of 90%. LN patients showed lower reactivity for all nine differentially reactive proteins (FIG. 3E). Pregnancy specific beta-1-glycoprotein 1 (PSG1), interferon regulatory factor 8 (IRF8), IL6R, myosin 7B (MYO7B), TPO, ITGA2B, polypyrimidine tract binding protein 1 (PTBP1) MYO1A and CD1d molecule (CD1D) had higher reactivity to the normal population. Using all nine proteins, a receiver operator curve was created with an area under the curve of 0.908, sensitivity of 90% and specificity of 80%.

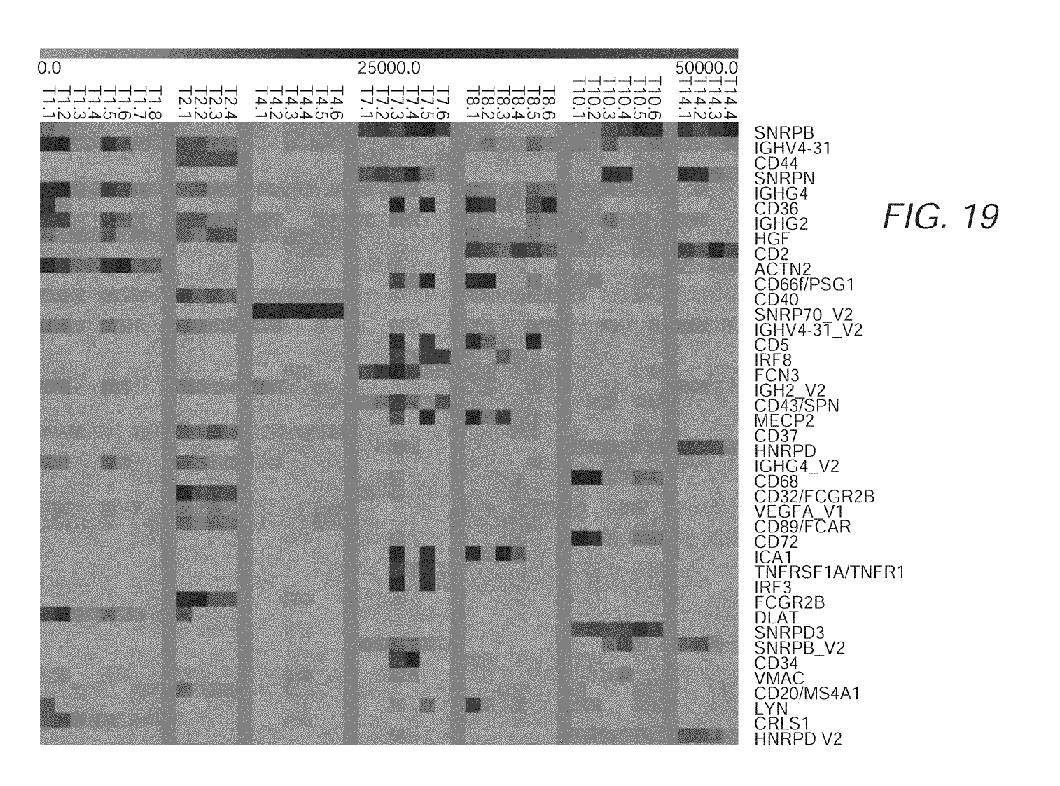

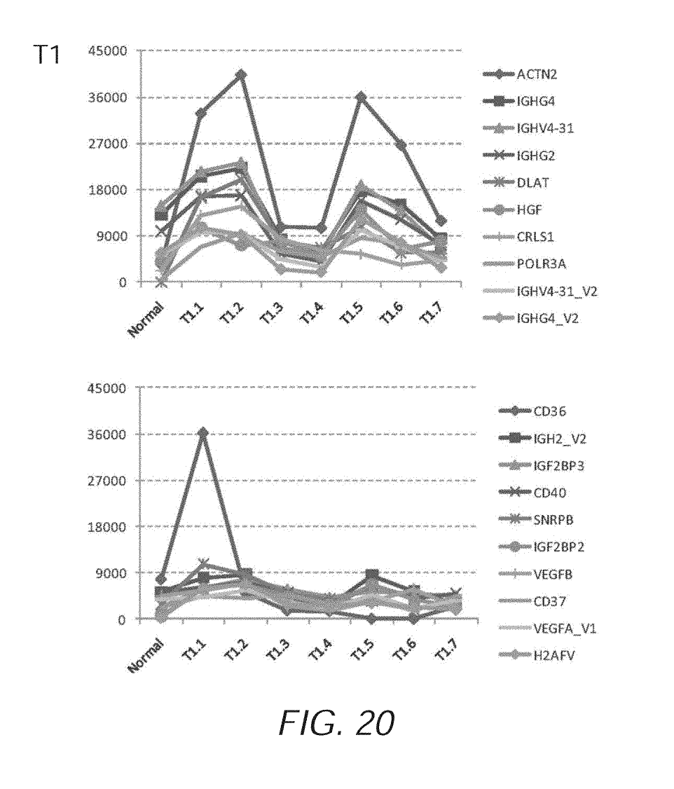

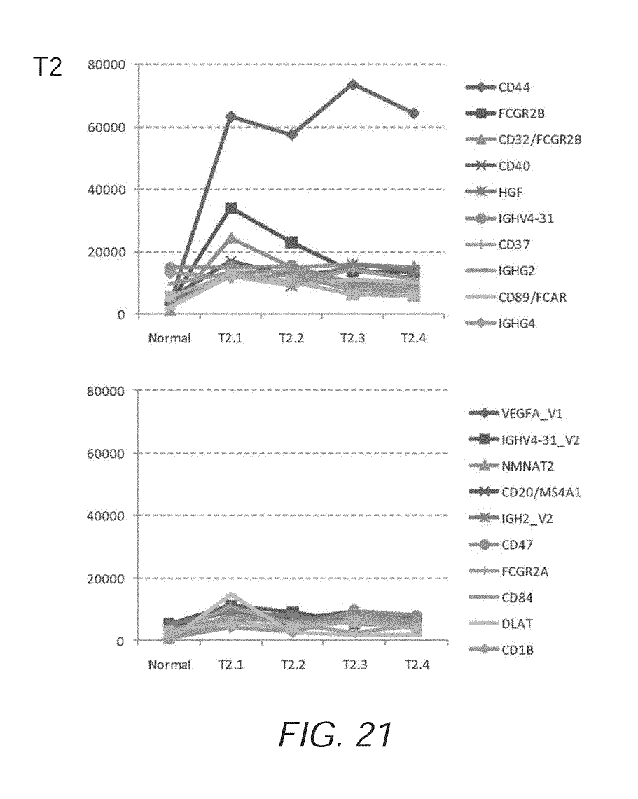

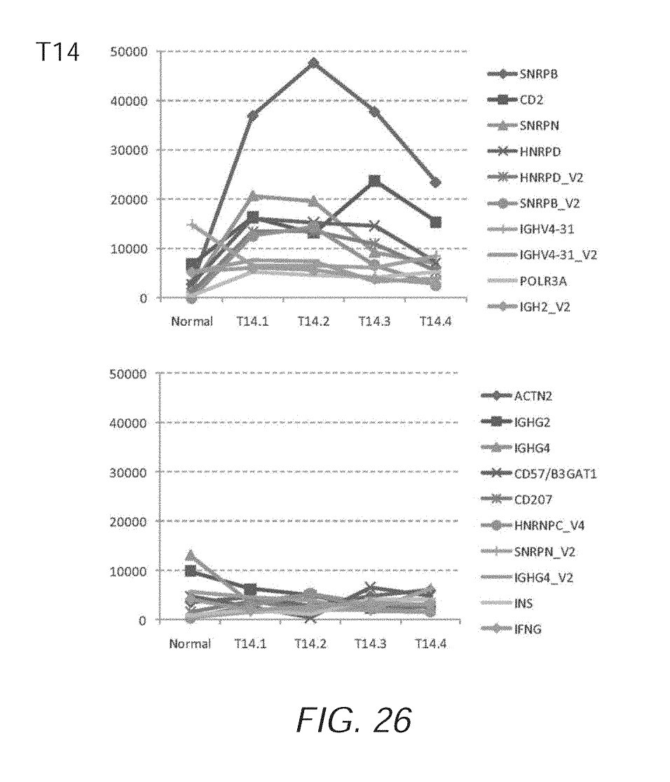

Serial bleeds for seven lupus nephritis (LN) patients that were undergoing treatment were probed on HA2. The serum samples were taken at different time points after treatment for LN had begun. The first time point in each of these serial bleeds, the “0.1” time points, were taken before treatment began. A heat map was created of the antigens that showed the most reactivity, and is shown in FIG. 19. FIGS. 20-26 show line graphs of the serial bleeds that were created from the data for each patient (Ti, T2, T4, T7, T8, T10, and T14). The mean values form the normal groups were used as a reference and as first point on each of the graphs.

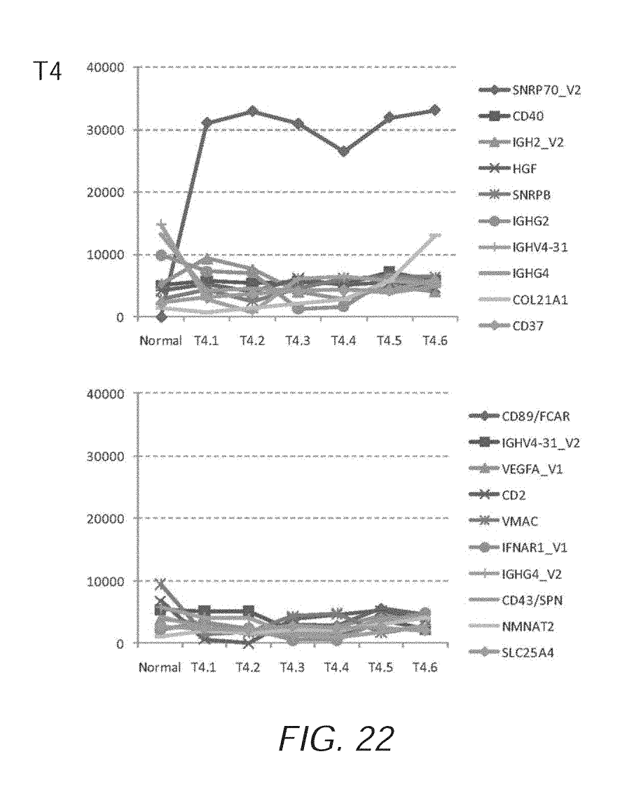

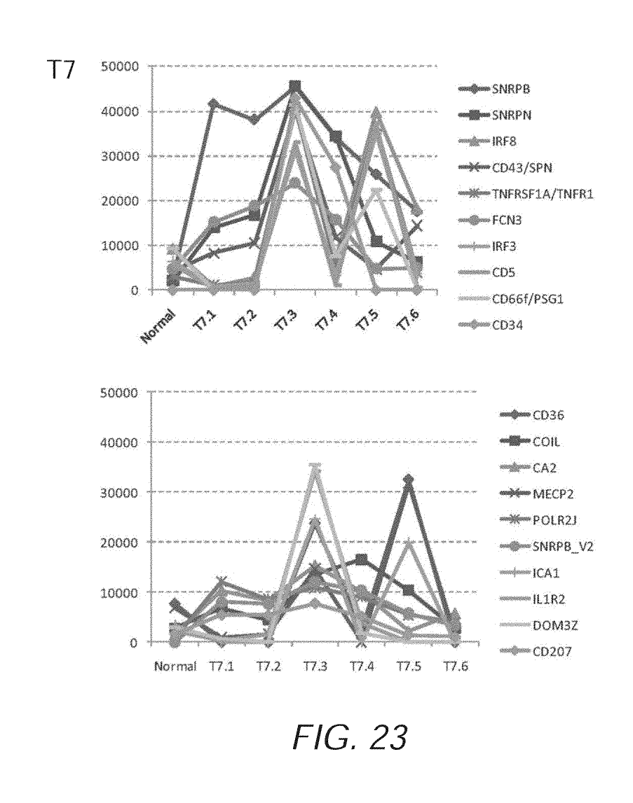

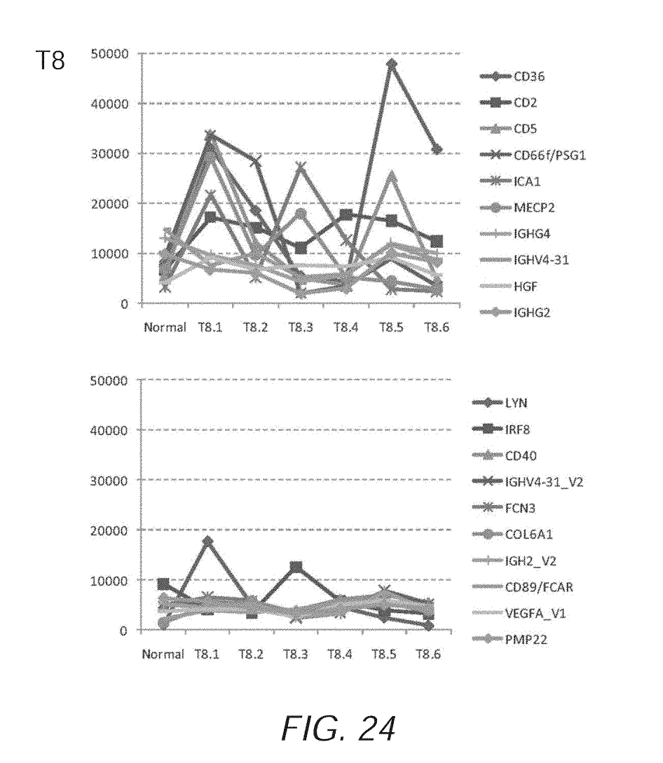

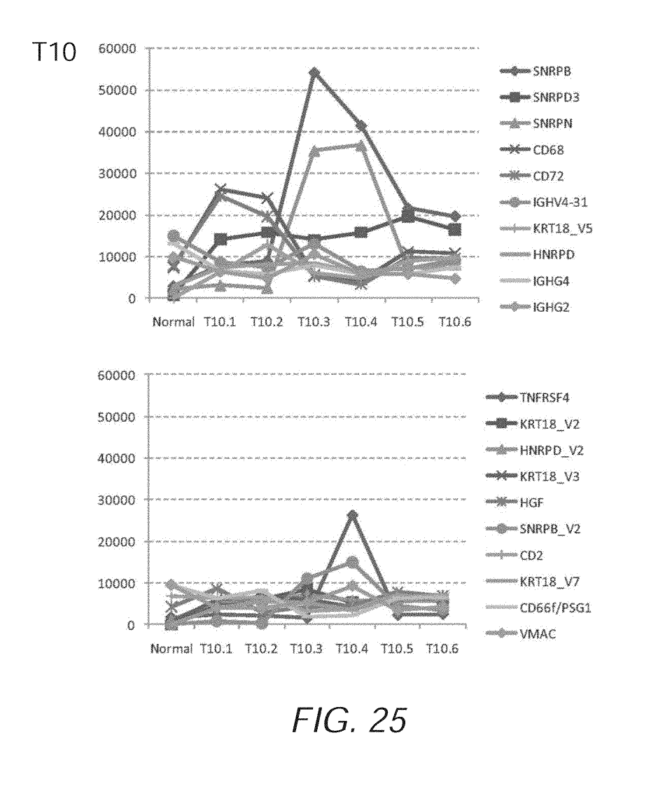

Much like the autoantibody profile at bleed 1, the changes seen in the subsequent bleeds appear very heterogeneous. The reactivity for patient Ti shows a downward trend from the first bleed. There is one antigen (actinin, alpha 2, ACTN2), however, that shows an substantial increase on bleeds 5 and 6 (FIG. 20). Patient T4 on the other hand is shows almost no change over the time course, with high reactivity to one antigen small nuclear ribonucleoprotein 70 kDa (SNRP70) and low reactivity to the other reactive antigens (FIG. 22). Patient T7 shows an increase of reactivity from the first three bleeds (FIG. 23). The most reactive proteins being SNRPB, PSG1, sialophorin (SPN), and CD34 molecule (CD34). Beginning from bleed four we see that individual auto-antigens behave differently. Some reactivities increase while some decrease. Patient T8 shows a general decrease in reactivity over time with a few spikes at different bleeds for different proteins (FIG. 24). Islet cell autoantigen 1, 69 kDa (ICA1) reactivity spiked at bleed 3, while CD36 reactivity spiked at bleed five and stayed elevated at bleed six. Patient T10, unlike the other four patients, started off with relatively low levels of reactivity. Reactivity to SNRPB and tumor necrosis factor receptor superfamily, member 4 (TNFRSF4) increased from bleed two to four, then settling back down to bleed one levels by bleed five (FIG. 25).

When compared to the mean values of the normal population, the first time point shows elevated antibody levels for some antigens, and baseline or slightly lower antibody levels for others. Each patient also showed a distinct antibody profile and time course signature. Thus, the data suggests that the biomarkers discovered using the ADI platform described herein have the potential to allow for personalized tracking of the efficacy of a treatment via the change in antibody levels against certain human proteins.

### EXAMPLE 3

### Breast Cancer Study

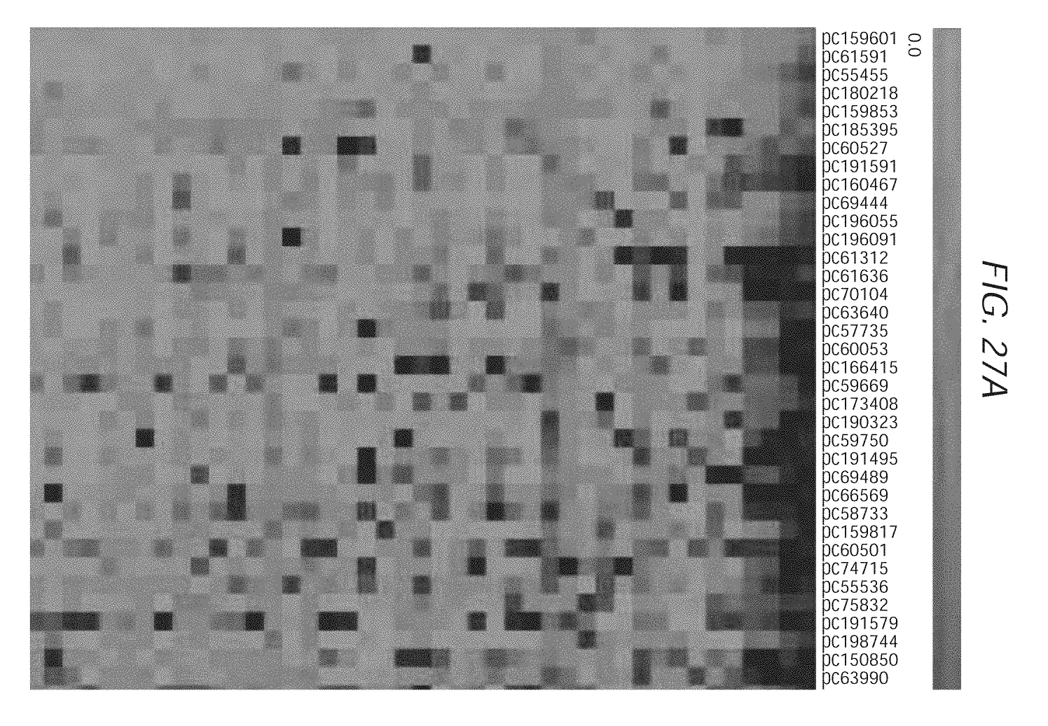

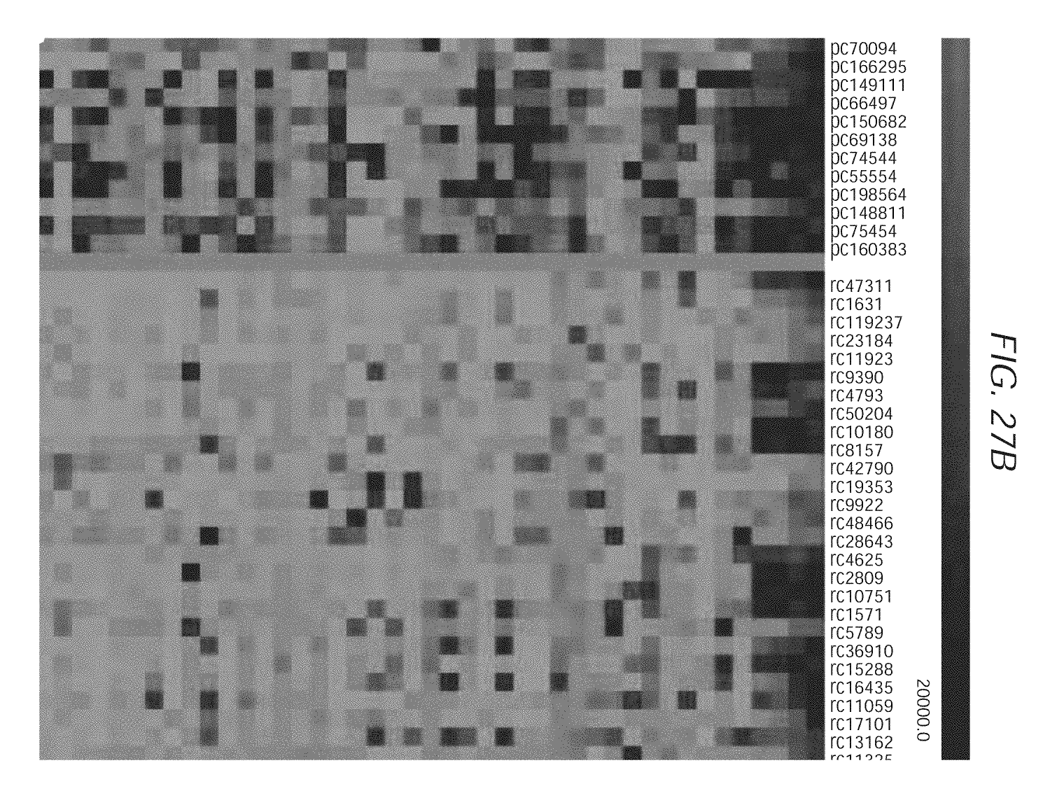

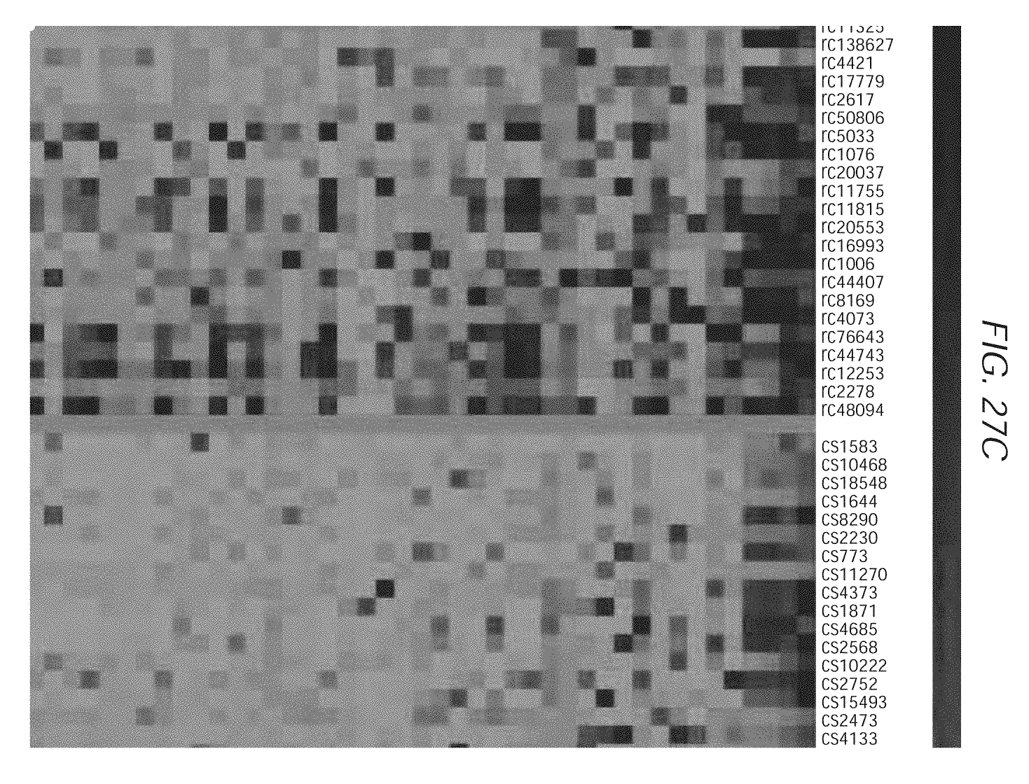

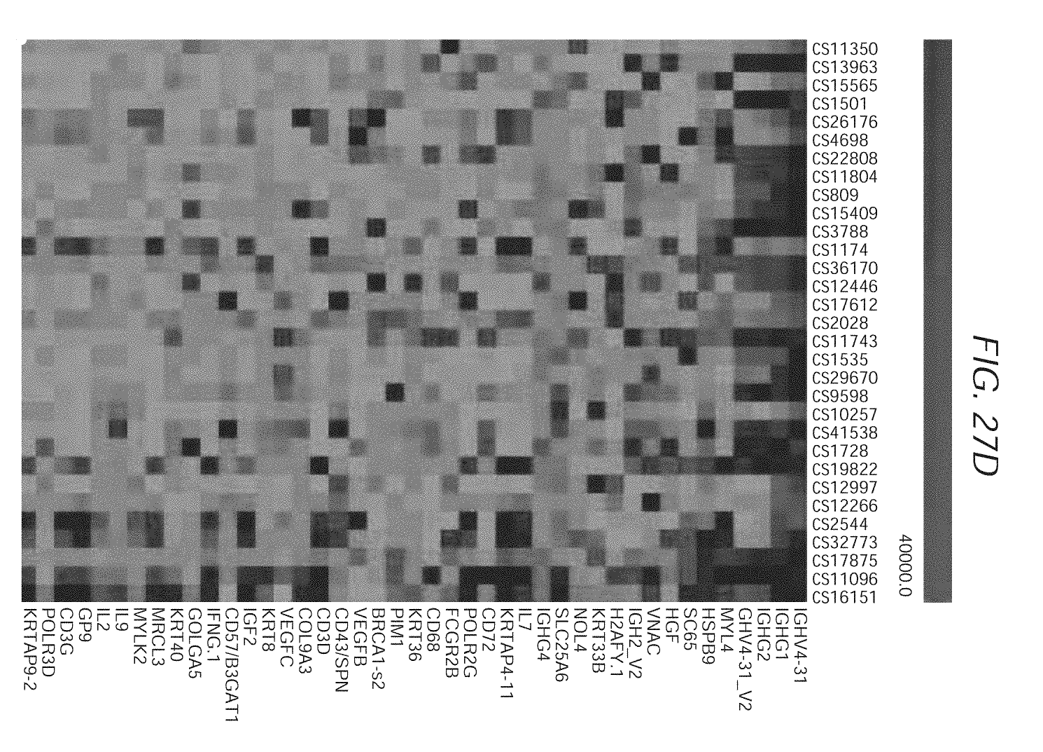

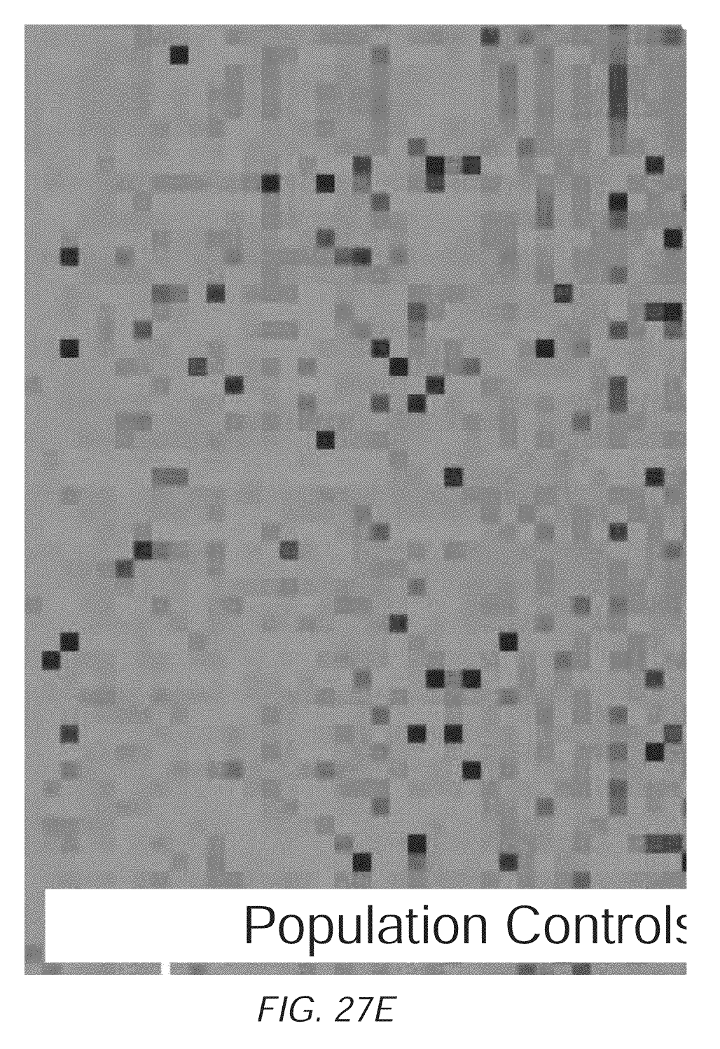

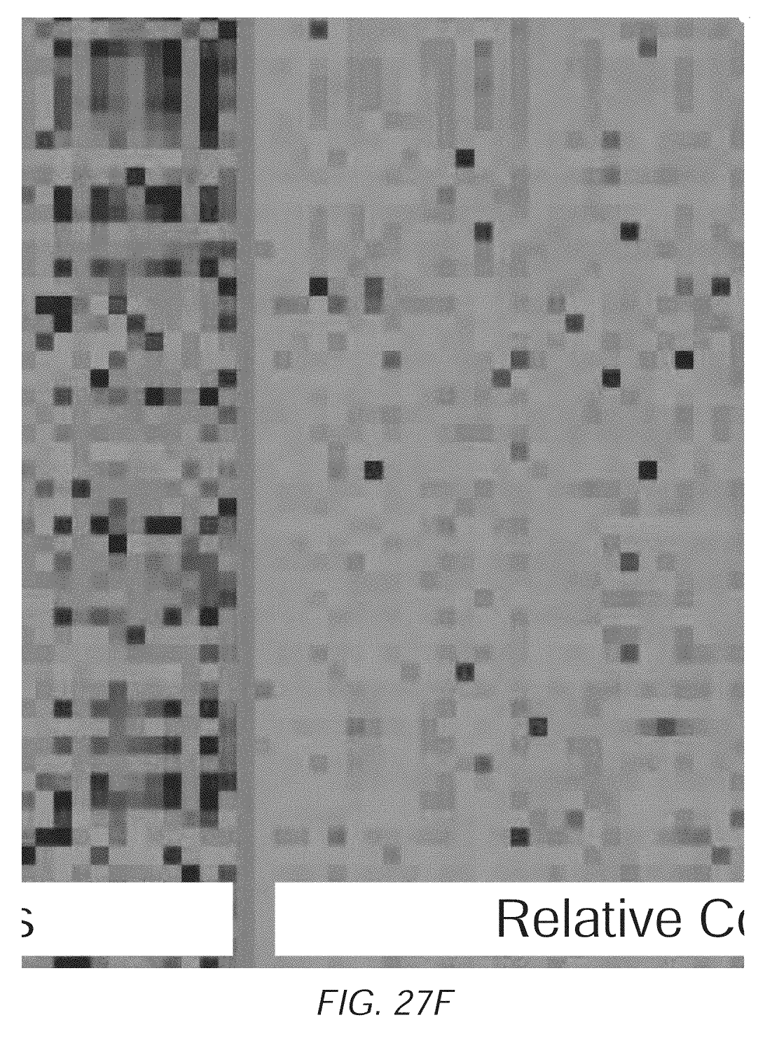

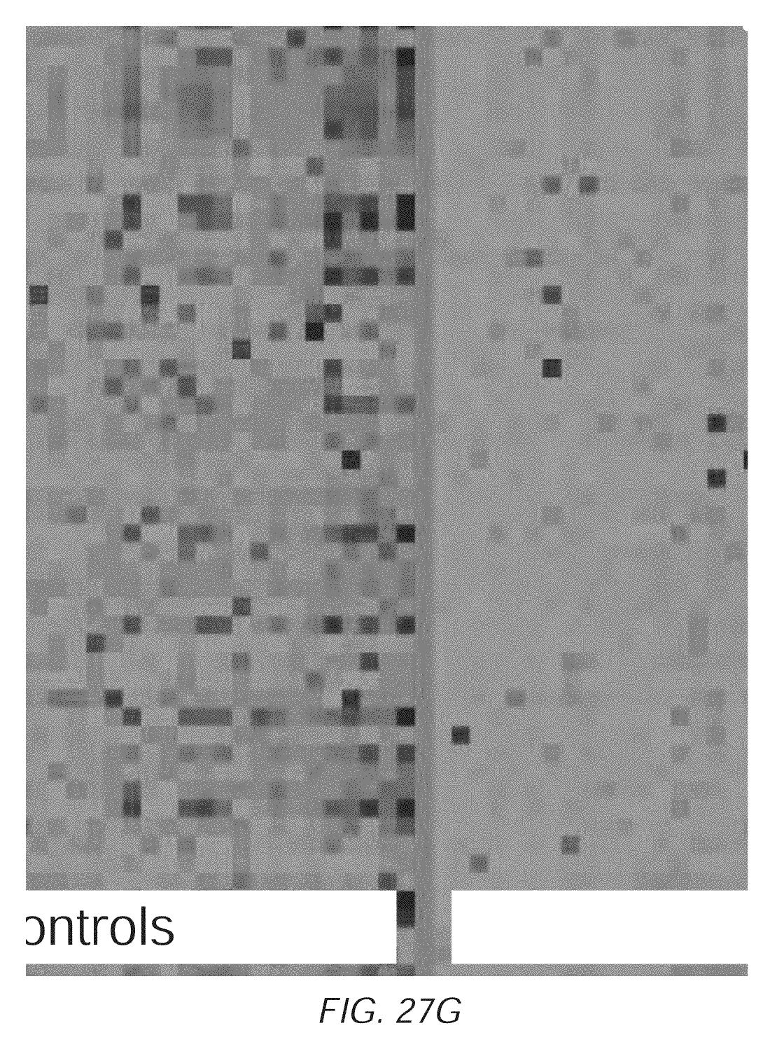

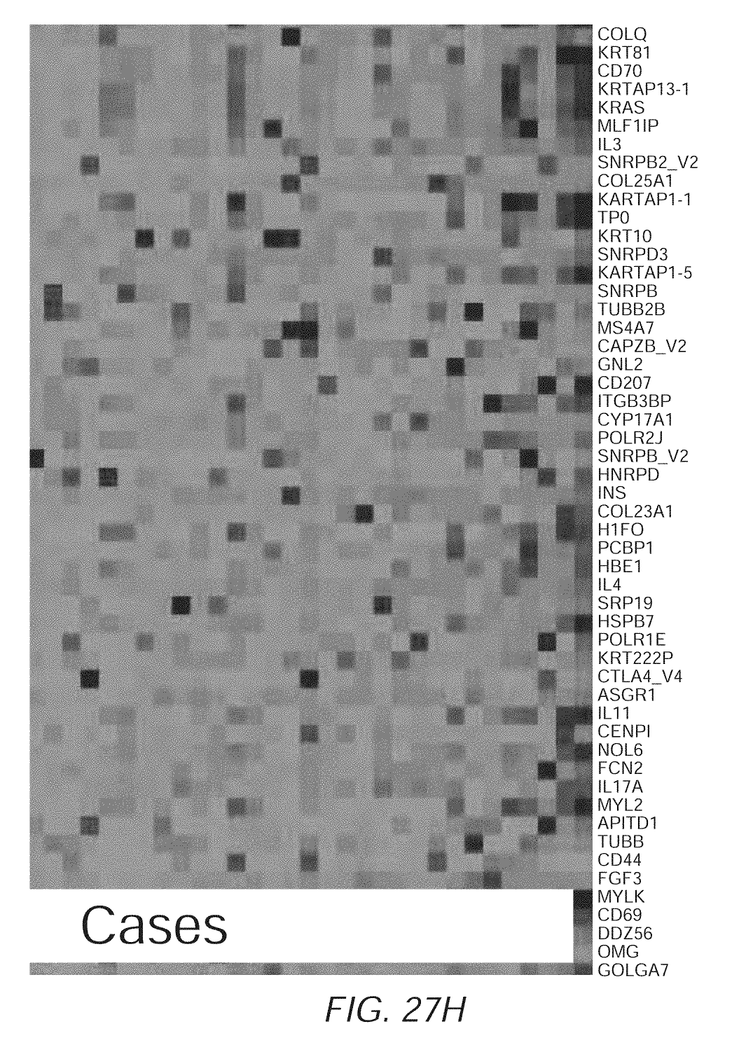

The HA2 chip was interrogated with serum samples from 48 breast cancer cases (CS), 48 blood-relative (sister) controls (RC), and 48 population controls (PC). Data was collected for the 144 serum samples for 840 proteins on the array using an IgG-specific secondary antibody to detect antibodies bound to the proteins. The HA2 chips were scanned and quantified using PerkinElmer ProscanArray Express™ v.4 software. The data from the mean-background columns was used to compile the raw data. The raw data was visualized in a heat map of the signal intensity data shown in FIGS. 27A-27H for 144 serum samples (columns) and the most reactive proteins on the chip (rows), which illustrates the autoantibody profile for patients breast cancer, their sister controls and population controls.

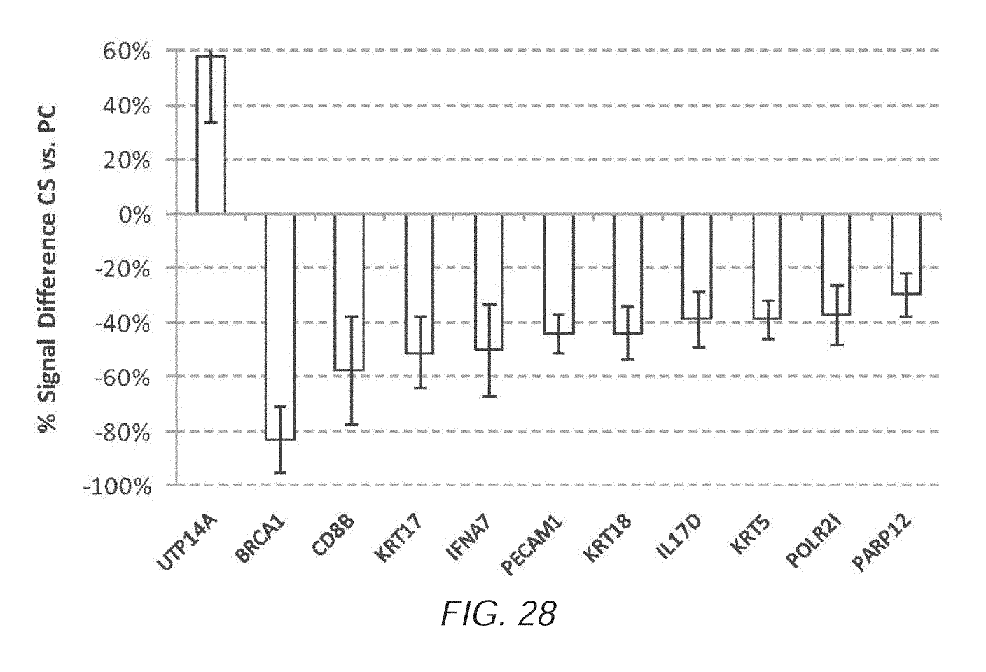

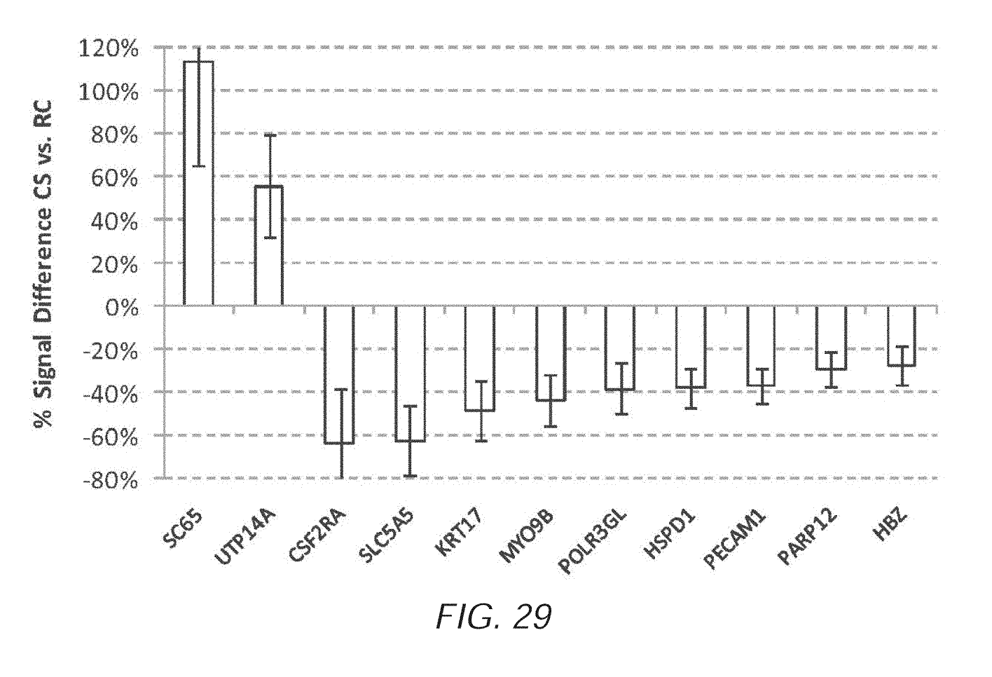

The compiled data was normalized by the application of VSN in R. The mean signal intensities, standard deviations, standard errors and the Bayesian t-test were also calculated in R. Using the control population as a baseline, the percentage change in the signal intensity for proteins with a p-value of less than 0.05 was assessed. When comparing the relative changes in signal intensities for the 11 proteins in the CS group compared with the PC as shown in FIG. 28, the biggest changes were in the antibodies against BRCA1 followed by UTP14 homolog A (UTP14A) and complement component 5a receptor 1 (C5AR1 or CD88). The mean signal intensities for BRCA1 and CD88 were found to be lower than the baseline, while UTP14A showed an increase in the CS group. FIG. 29 illustrates changes in signal intensities for the 11 proteins in the CS group compared with the RC. Similarly, the CS versus RC comparison showed increased levels of UTP14A. The largest increase, however, was for the known autoantigen synaptonemal complex protein SC65 (SC65). Nine other proteins showed lower signal intensities for the CS than for the RC.

### EXAMPLE 4

### Use of a High-Throughput Proteome Microarray to Identify Autoantibody Signatures in Patients with Systemic Lupus Erythematosus

Systemic lupus erythematosus (SLE/lupus) is an autoimmune disease with a complex etiopathology. Diagnosis is often difficult and management of the numerous clinical manifestations can be problematic, even for experienced clinicians. Serologically, it is characterized by autoantibodies to a diverse range of human proteins. Monitoring these antibodies, particularly specificity and titers, has been a mainstay of diagnosis and disease management for decades. However autoantibody measurement has never been entirely satisfactory for providing warnings of disease flares or organ involvement.

However, the use of serological methods remains attractive because they are relatively non-invasive and can be performed quickly. To that end, the inventors have developed a high-throughput proteomic microarray platform that allows thousands of protein gene products or antigens to be printed on a glass slide and used to interrogate sera from humans or animals (e.g., Molina D M, Morrow, W. J. W., Liang X L. Use of high-throughput proteomic microarrays for the discovery of disease-associated molecules. In Biomarkers in Drug Development: a handbook of practice, application and strategy. Eds. Bleavins M, Carini, C, Jurima-Romet, M, Rahbari, R. 2010. Wiley (New York) 2010). The arrays can advantageously be produced very quickly, and have been used with considerable success to identify diagnostic and vaccine candidates in a number of pathogen systems including, tuberculosis (e.g., Kunnath-Velayudhan S, Salamon H, Wang H Y, Davidow A L, Molina D M, Huynh V T, Cirillo D M, Michel G, Talbot E A, Perkins M D, Felgner P L, Liang X, Gennaro M L. 2010. Dynamic antibody responses to the Mycobacterium tuberculosis proteome. Proc Natl Acad Sci USA. 107(33):14703-8), brucellosis (e.g., Liang L, Leng D, Burk C, Nakajima-Sasaki R, Kayala M, Atluri V L, Pablo J, Unal B, Ficht T A, Gotuzzo E, Saito M, Morrow W J W, Liang X, Baldi P, Vinetz J, Felgner P L, Tsolis R M. 2010. Large scale immune profiling of infected humans and goats reveals differential recognition of Brucella melitensis antigens. PLoS Negl Trop Dis. 4(5):e673), Chlamydia (e.g., Molina D M, Pal S, Kayala M A, Teng A, Kim P J, Baldi P, Felgner P L, Liang X, de la Maza L M. 2009. Identification of immunodominant antigens of Chlamydia trachomatis using proteome microarrays. Vaccine 28 (17):3014-24), Lyme disease (e.g., Barbour A G, Jasinskas A, Kayala M A, Davies D H, Steere A C, Baldi P, Felgner P L. 2008. A genome-wide proteome array reveals a limited set of immunogens in natural infections of humans and white-footed mice with Borrelia burgdorferi. Infect Immun. 76(8):3374-89), as well as identify new targets of pemphigus auto-antibodies (e.g., Kalantari-Dehagi M, Molina D M, Farhadieh M, Morrow W J W, Liang X, Felgner P L, Grando S A. New targets of pemphigus vulgaris antibodies identified by protein array technology. Exp Dermatol. 20(2):154-6).

Sera were studied from patients attending the autoimmune rheumatic disease clinic at the University College Hospital over the past 25 years. All patients with lupus met the revised criteria of the American College of Rheumatology. Those considered to have kidney involvement had to have had a confirmatory biopsy. Patients with Sjögren's syndrome met the American European Consensus Criteria. Those with myositis had three out of the four of the criteria proposed by Bohan and Peter and those with rheumatoid arthritis all had four or more of the revised criteria of the American Rheumatism Association.

Human protein microarray chips were fabricated in the manner described above. The Human protein microarray chips were probed with human sera from systemic lupus erythomatosis, lupus nephritis, polymyositis, rheumatoid arthritis, scleroderma and Sjögrens's Syndrome patients, as well as age, sex, ethnicity matched normal healthy control sera. Prior to microarray probing, the sera were diluted to 1/100 in Protein Array Blocking Buffer (Whatman) containing E. coli lysate at a final concentration of 10%, approximately 1-2 mg/ml, and incubated for 30 minutes at room temperature while mixing. The microarrays were blocked using 1×-blocking buffer for 30 minutes while the serum samples were pre-incubating. The blocking buffer was removed and the diluted serum was added to the microarrays and hybridized overnight in a humidified box. Following washing, the slides were incubated with diluted biotinlyated goat anti-human IgG (H+L) (JacksonImmuno Research Laboratories Inc.™ of West Grove, Pa.) for one hour at room temperature with agitation. Following washing, bound antibodies were detected by incubation with streptavidin-conjugated Sensilight P3 (Columbia Biosciences™). Following washing and drying overnight, intensities were quantified using QuantArray™ software. Microarrays were scanned, quantified, and all signal intensities were corrected for background.

The statistical analysis was performed as previously described. Briefly, the data was calibrated and transformed using the VSN package in the R statistical environment. Differential reactivity analysis was then performed using Bayes-regularized t-tests. To address multiple comparisons, p-values were adjusted using the Benjamini-Hochberg procedure for controlling the Family Wise Error Rate (FWER). All reported p-values are Benjamini-Hochberg corrected unless otherwise noted. Finally, the data was retransformed into an approximate raw scale by taking the base 2 anti-log of the values for bar plot visualizations.

The antigens were ranked by their adjusted Benjamin-Hochberg p-values. Each antigen could serve as a single marker. A ROC curve analysis was performed to each of the antigens. From statistical literature, it is known that combining multiple markers increases the accuracy measured by the area under the ROC curve (AUROC). See, e.g., Su JQ and Liu J (1993). Linear combination of multiple diagnostic markers. Journal of American Statistical Association 88, 1350-1355 and Pepe M S and Thompson M L (2000). Combining diagnostic test results to increase accuracy. Biostatistics 1(2): 123-140. Optimal linear combination (OLC) was used to progressively combine the top discriminating antigens, and the AUROC of each OLC was plotted with progressively increased number of antigens and the graph usually plateaued after certain number of antigens. That means it does not increase the accuracy of the combined marker by adding more antigens. Then the selected antigens are used for the final OLC. The ROC curve analysis was performed using the R packages ROCR and ROC which produces the empirical ROC curve, an estimate of the AUROC and a list of cut points and corresponding sensitivities and specificities. The optimal cut point was selected to be the closest to the point of (0,1), which is the accuracy for a gold standard.

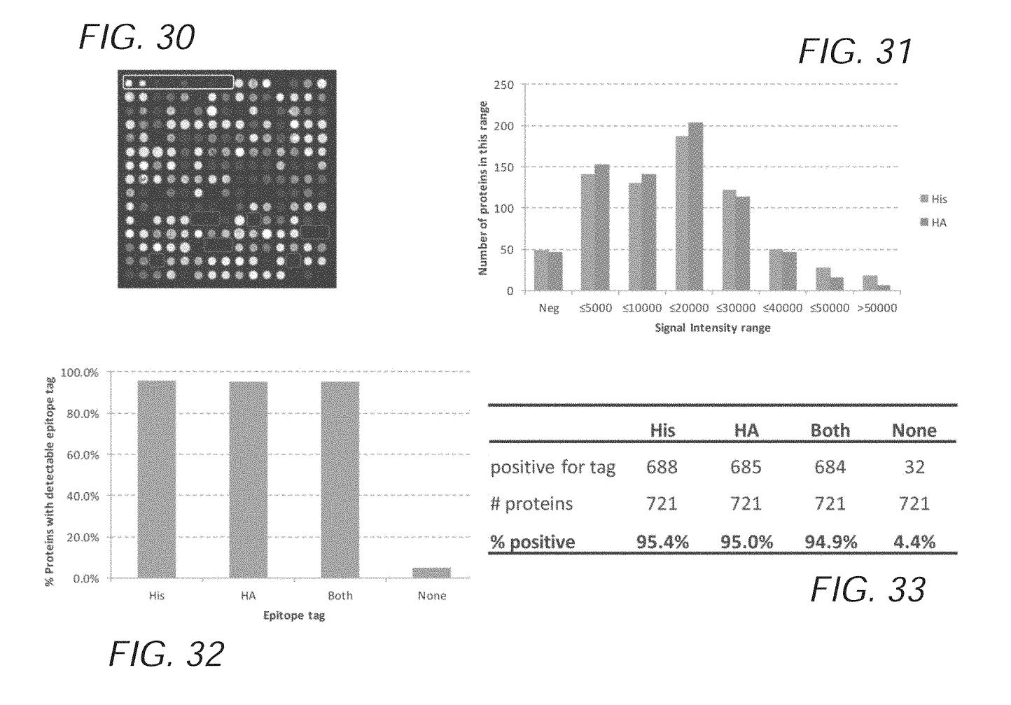

A human autoimmune-associated protein (HAAP) chip was composed of 713 total human proteins, representing proteins identified as described above and their splice and/or cDNA variants. Only 48 clones were negative for cloning and sequencing. Once expressed and arrayed, the chips were probed with anti-polyHistidine and anti-HA antibodies to verify the expression of the proteins as a quality control (QC) method. The chips were scanned and quantified using PerkinElmer Proscan Array Express™ v.4 software. The data from the mean-background columns was used to compile the raw data. FIG. 30 shows an image of one sub-array (out of 4) representing approximately 207 different expression products and 18 control spots visualized using the C-terminal HA tag and the anti-HA antibody. FIG. 31 shows the distribution of the mean signal intensities for the QC probing, while FIGS. 32 and 33 show that greater than 95% of the expression products were recognized via the detection of one or the other tag.

Normal Controls have Circulating Antibodies Against Human Proteins

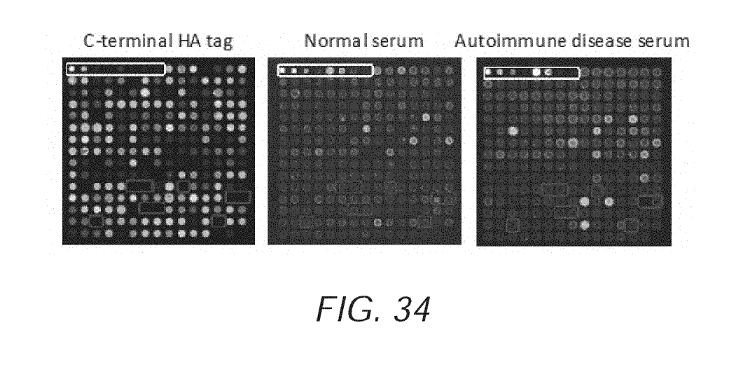

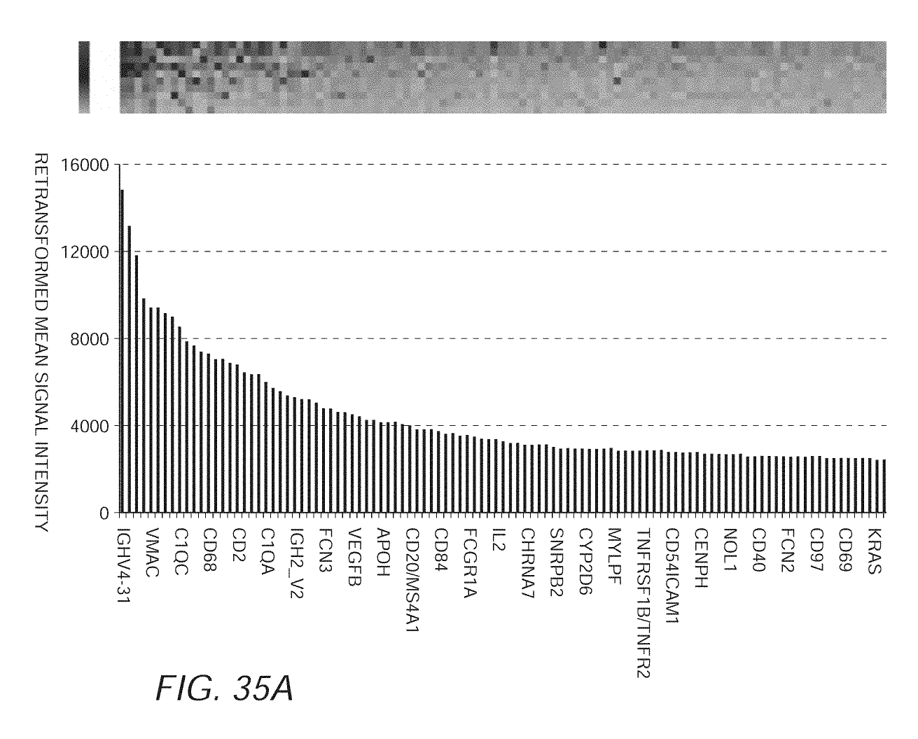

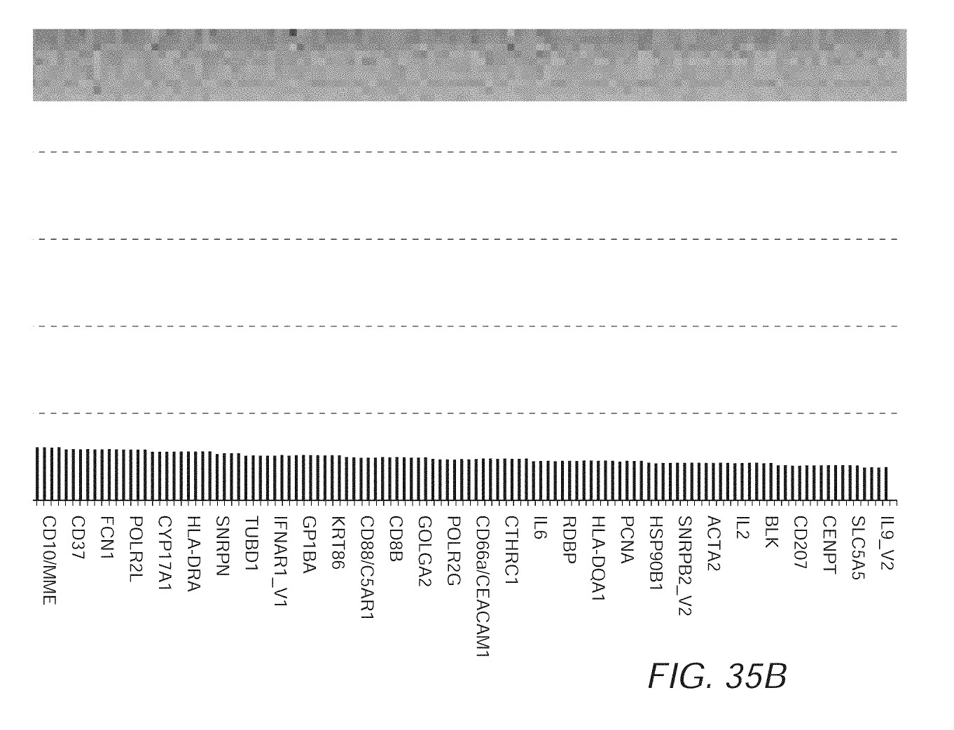

Serum from 10 normal donors was probed on the HAAP chip to establish a baseline for the subsequent probing of lupus patient sera. Interestingly, the normal controls were found to have circulating auto-antibodies against proteins on the chip. FIG. 34 is a heat map with the individual normal donors (rows) and the proteins (columns), which shows a cluster of reactivity towards the left side of the heat map as well as more heterogeneously distributed reactivity to proteins on the right side of the heat map. The mean signal intensities for the proteins were tabulated from the normalized data and the values plotted in a histogram shown in FIGS. 35A-35B. 225 proteins or auto-antigens were recognized by the auto-antibodies in the serum of normal donors.

Profiling Lupus

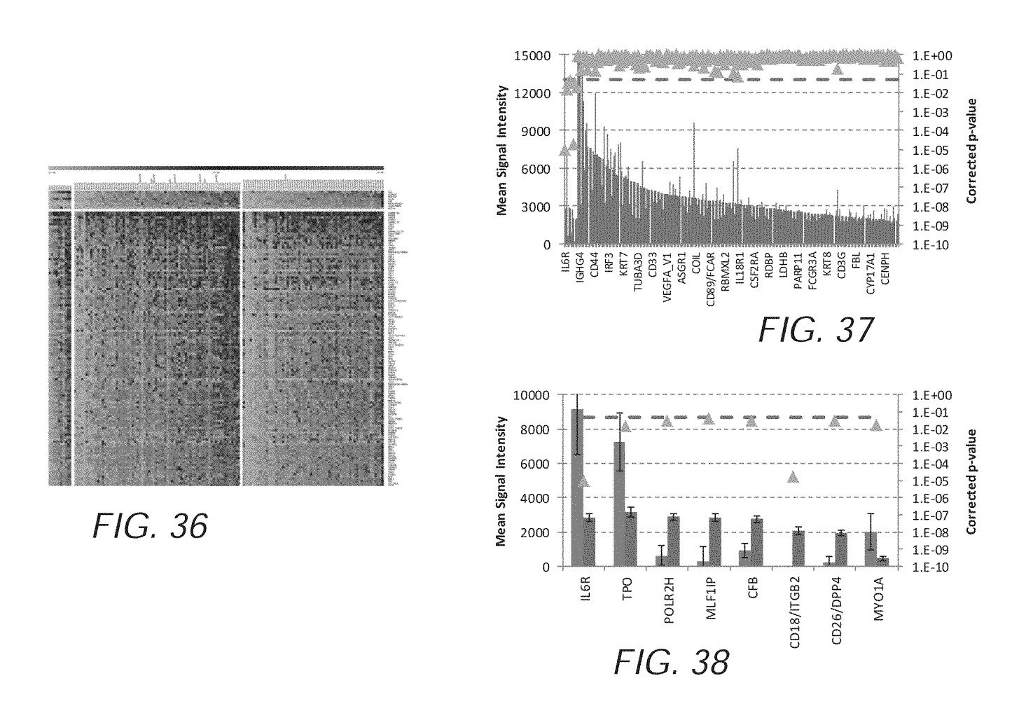

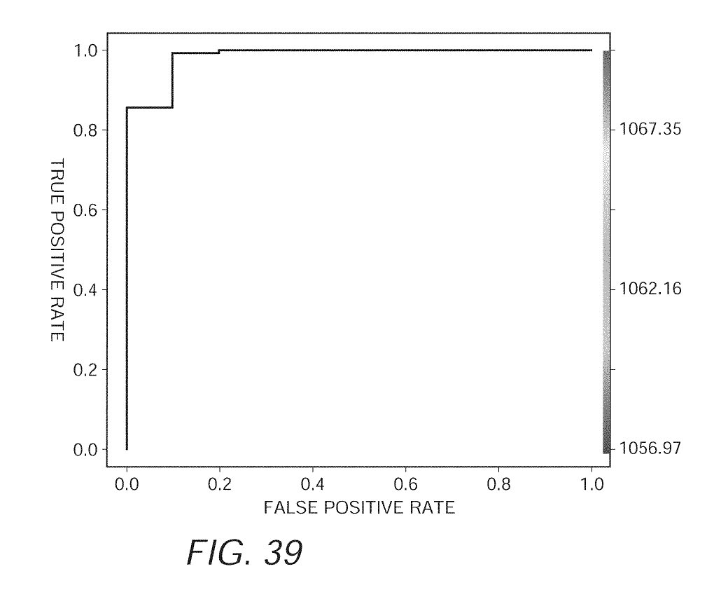

Serum from 133 lupus patients was probed on the HAAP chip. The data collected was merged with the data for normal. The entire data set was normalized and the auto-antibody profile of the lupus patients was compared with that of the normal. A heat map shown in FIG. 36 was created to examine the reactivity pattern of the 143 serum samples. FIG. 37 illustrates a histogram of all the reactive proteins. To tease out the disease biomarkers, the raw data was normalized using VSN. Using the normalized data, the mean signal intensities, the standard deviation and standard errors were calculated for each group of samples in the statistical environment known as R. To determine which antigens were potential biomarkers, the disease groups were compared with the normal group using Benjamini-Hochberg corrected p-values (BHp) calculated from Bayesian regularized t-tests performed in R. As shown in FIGS. 36-38, there is a small subset of auto-antigens that show different reactivities profiles in the lupus patients from the normal donors, while there are a large number of proteins that are reactive in both groups. There are eight differentially reactive proteins for which the mean intensity, standard error and BHp were plotted. When looking at autoantibodies, there are two potential outcomes of human disease: an increase in circulating antibodies or a decrease in existing antibodies in response to disease pathology. The former is seen for five proteins: polymerase (RNA) II (DNA directed) polypeptide H (POLR2H), MLF1 interacting protein (MLF1IP), complement factor B (CFB), integrin beta 2 (complement component 3 receptor 3 and 4, ITGB2), and dipeptidyl-pepsidase 4 (DPP4). The latter is seen for Interleukin 6 receptor (IL6R), thrombopoietin (TPO) and myosin 1A (MYO1A). Using all eight proteins, a receiver operator curve is created shown in FIG. 39 with and area under the curve of 0.986, sensitivity of 99% and specificity of 90%.

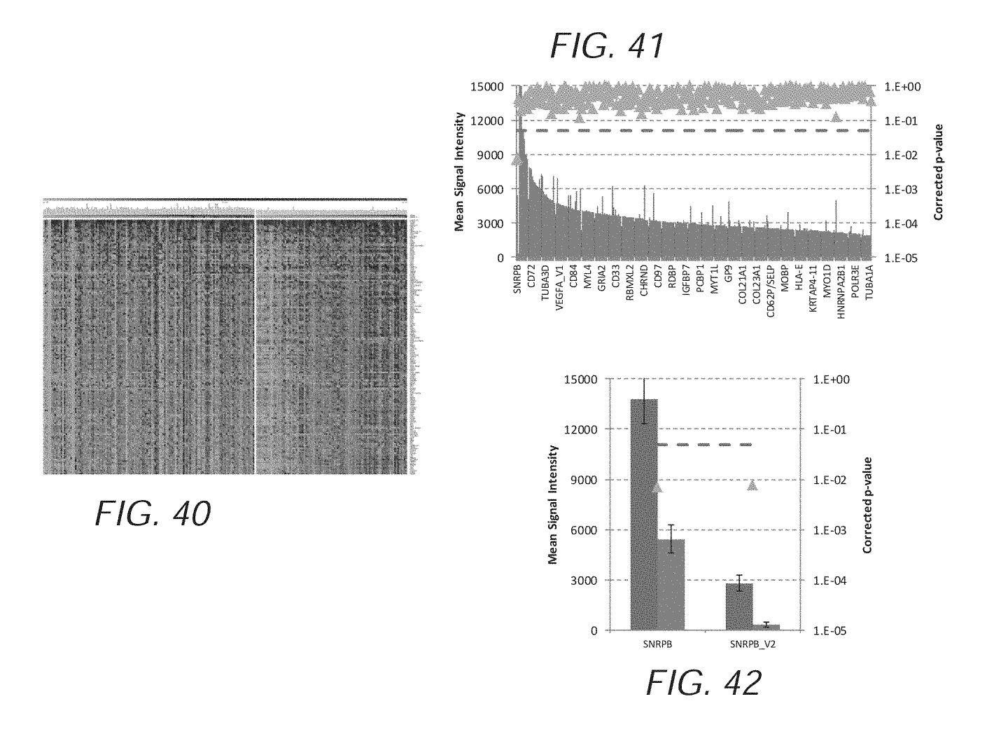

Serum from 95 patients with polymyositis, rheumatoid arthritis, scleroderma and Sjögren's syndrome were also probed to be used as autoimmune disease controls to determine whether or not we could identify lupus specific auto-antibodies. As shown in FIGS. 40-42, two versions of the same protein, protein small nuclear ribonucleoprotein polypeptides B and B1 (SNRPB), have higher reactivity in the lupus group than in the disease controls.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An antigen composition, comprising:
a plurality of autoantibody reactive antigens associated with a carrier;
wherein at least two of the plurality of antigens have quantified and known relative autoantibody reactivities with respect to sera of a population affected by a disease;
wherein the at least two antigens have a known association with a disease parameter; and
wherein the plurality of antigens are selected from a group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, UTP14a, DPP4, IL6R, ITGB2, MLF1IP, MYO1A, POLR2H, CD1D, IRF8, ITGA2B, MYO7B, PSG1, PTBP1, CD1C, CD46, CENPQ, CFB, HLA-DQB1, KRTAP9-3, MYT1L, SLC7A5, TPO, CD14, CD55, COL9A1, COLQ, DLAT, FGF7, H3F3B, IL1RAPL2, IL8, PADI4, PIP4K2C, PLAUR, STK19, APOH, BANK1, BLK, CD3E, CD70, CD80, CD86, CEACAM6, CEACAM8, CENPT, COL1A2, DDC, FCGR1A, H2AFX, H2AFY, HBA1, HBA2, HBD, HSP90B1, HSPB7, IGHG2, IGHG4, IGHM, IGHV4-31, IL6, ITGB3BP, KRTAP13-1, MBP, MOBP, MS4A8B, MYH9, MYO1D, NMNAT2, NOL1, PDCD1, POLR2C, POLR2J2, POLR3D, PSIP1, SRP19, STAT4, CALR3, CD34, CD69, CD93, CENPA, CHRNA1, COL20A1, COL4A6, FCGR3A, H1F0, HBM, HLA-C, HLA-F, IFNG, IGFL2, IGH2, IGHV7-81, KRT73, KRT19, KRTAP9-8, NOLA3, POLR3H, and UEVLD, or fragments thereof

2. The antigen composition of claim 1, wherein the disease is breast cancer, and wherein the plurality of antigens are selected from the group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, and UTP14a, or fragments thereof.

3. The antigen composition of claim 1, wherein the disease is lupus, and wherein the plurality of antigens are selected from the group consisting of DPP4, IL6R, ITGB2, MLF1IP, MYO1A, POLR2H, and TPO, or fragments thereof.

4. The antigen composition of claim 1, wherein the disease is lupus nepritis, and wherein the plurality of antigens are selected from the group consisting of CD1D, IL6R, IRF8, ITGA2B, MYO1A, MYO7B, PSG1, PTBP1, and TPO, or fragments thereof.

5. The antigen composition of claim 1, wherein the disease is systemic lupus erythematosus, and wherein the plurality of antigens are selected from the group consisting of CD1C, CD46, CENPQ, CFB, DPP4, HLA-DQB1, IL6R, ITGB2, KRTAP9-3, MLF1IP, MYT1L, POLR2H, SLC7A5, and TPO, or fragments thereof.

6. The antigen composition of claim 1, wherein the disease is polymyositis, and wherein the plurality of antigens are selected from the group consisting of CD14, CD1C, CD46, CD55, CFB, COL9A1, COLQ, DLAT, DPP4, FGF7, H3F3B, IL1RAPL2, IL6R, IL8, ITGB2, KRTAP9-3, MLF1IP, MYT1L, PADI4, PIP4K2C, PLAUR, POLR2H, POLR2I, PSG1, SLC7A5, and STK19, or fragments thereof.

7. The antigen composition of claim 1, wherein the disease is rheumatoid arthritis, and wherein the plurality of antigens are selected from the group consisting of APOH, BANK1, BLK, CD1C, CD14, CD3E, CD70, CD80, CD86, CEACAM6, CEACAM8, CENPT, CFB, COL1A2, DDC, DPP4, FCGR1A, H2AFX, H2AFY, H3F3B, HBA1, HBA2, HBD, HLA-DQB1, HSP90B1, HSPB7, IGHG2, IGHG4, IGHM, IGHV4-31, IL12A, IL6, IL6R, ITGB3BP, KRTAP13-1, KRTAP9-3, MBP, MLF1IP, MOBP, MS4A8B, MYH9, MYO1D, MYT1L, NMNAT2, NOL1, PDCD1, PIP4K2C, POLR2C, POLR2H, POLR2I, POLR2J2, POLR3D, PSIP1, SRP19, STAT4, and STK19, or fragments thereof.

8. The antigen composition of claim 1, wherein the disease is scleroderma, and wherein the antigen is IL6R, or a fragment thereof.

9. The antigen composition of claim 1, wherein the disease is Sjögren's syndrome, and wherein the plurality of antigens are selected from the group consisting of APOH, CALR3, CD1C, CD14, CD34, CD3E, CD46, CD69, CD93, CEACAM8, CENPA, CENPQ, CFB, CHRNA1, COL20A1, COL4A6, DPP4, FCGR3A, H1F0, H2AFX, H3F3B, HBA1, HBA2, HBD, HBM, HLA-C, HLA-DQB1, HLA-F, HSPB7, IFNG, IGFL2, IGH2, IGHV7-81, IL1RAPL2, IL6R, ITGB2, keratin 73, KRT19, KRTAP9-3, KRTAP9-8, MBP, MLF1IP, MYT1L, NOLA3, POLR2H, POLR2I, POLR3D, POLR3H, PTBP1, STK19, and UEVLD, or fragments thereof.

10. A method of predicting a likelihood of a patient having a disease, comprising:
determining autoantibody reactivity against one or more antigens, or their variants, in a serum sample obtained from a patient;
wherein the one or more antigens are selected from a group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, UTP14a, DPP4, IL6R, ITGB2, MLF1IP, MYO1A, POLR2H, CD1D, IRF8, ITGA2B, MYO7B, PSG1, PTBP1, CD1C, CD46, CENPQ, CFB, HLA-DQB1, KRTAP9-3, MYT1L, SLC7A5, TPO, CD14, CD55, COL9A1, COLQ, DLAT, FGF7, H3F3B, IL1RAPL2, IL8, PADI4, PIP4K2C, PLAUR, STK19, APOH, BANK1, BLK, CD3E, CD70, CD80, CD86, CEACAM6, CEACAM8, CENPT, COL1A2, DDC, FCGR1A, H2AFX, H2AFY, HBA1, HBA2, HBD, HSP90B1, HSPB7, IGHG2, IGHG4, IGHM, IGHV4-31, IL6, ITGB3BP, KRTAP13-1, MBP, MOBP, MS4A8B, MYH9, MYO1D, NMNAT2, NOL1, PDCD1, POLR2C, POLR2J2, POLR3D, PSIP1, SRP19, STAT4, CALR3, CD34, CD69, CD93, CENPA, CHRNA1, COL20A1, COL4A6, FCGR3A, H1F0, HBM, HLA-C, HLA-F, IFNG, IGFL2, IGH2, IGHV7-81, KRT73, KRT19, KRTAP9-8, NOLA3, POLR3H, and UEVLD, or fragments thereof; and
wherein autoantibody reactivity against the one or more antigens indicates an increased likelihood of the patient having a disease.

11. The method of claim 10, wherein the disease is breast cancer, and wherein the plurality of antigens are selected from the group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, and UTP14a, or fragments thereof.

12. The method of claim 10, wherein the disease is lupus, and wherein the plurality of antigens are selected from the group consisting of DPP4, IL6R, ITGB2, MLF1IP, MYO1A, POLR2H, and TPO, or fragments thereof.

13. The method of claim 10, wherein the disease is lupus nephritis, and wherein the plurality of antigens are selected from the group consisting of CD1D, IL6R, IRF8, ITGA2B, MYO1A, MYO7B, PSG1, PTBP1, and TPO, or fragments thereof

14. The method of claim 10, wherein the disease is systemic lupus erythematosus, and wherein the plurality of antigens are selected from the group consisting of CD1C, CD46, CENPQ, CFB, DPP4, HLA-DQB1, IL6R, ITGB2, KRTAP9-3, MLF1IP, MYT1L, POLR2H, SLC7A5, and TPO, or fragments thereof.

15. The method of claim 10, wherein the disease is polymyositis, and wherein the plurality of antigens are selected from the group consisting of CD14, CD1C, CD46, CD55, CFB, COL9A1, COLQ, DLAT, DPP4, FGF7, H3F3B, IL1RAPL2, IL6R, IL8, ITGB2, KRTAP9-3, MLF1IP, MYT1L, PADI4, PIP4K2C, PLAUR, POLR2H, POLR2I, PSG1, SLC7A5, and STK19, or fragments thereof.

16. The method of claim 10, wherein the disease is rheumatoid arthritis, and wherein the plurality of antigens are selected from the group consisting of APOH, BANK1, BLK, CD1C, CD14, CD3E, CD70, CD80, CD86, CEACAM6, CEACAM8, CENPT, CFB, COL1A2, DDC, DPP4, FCGR1A, H2AFX, H2AFY, H3F3B, HBA1, HBA2, HBD, HLA-DQB1, HSP90B1, HSPB7, IGHG2, IGHG4, IGHM, IGHV4-31, IL12A, IL6, IL6R, ITGB3BP, KRTAP13-1, KRTAP9-3, MBP, MLF1IP, MOBP, MS4A8B, MYH9, MYO1D, MYT1L, NMNAT2, NOL1, PDCD1, PIP4K2C, POLR2C, POLR2H, POLR2I, POLR2J2, POLR3D, PSIP1, SRP19, STAT4, and STK19, or fragments thereof.

17. The method of claim 10, wherein the disease is scleroderma, and wherein the antigen is IL6R, or a fragment thereof.

18. The method of claim 10, wherein the disease is Sjögren's syndrome, and wherein the plurality of antigens are selected from the group consisting of APOH, CALR3, CD1C, CD14, CD34, CD3E, CD46, CD69, CD93, CEACAM8, CENPA, CENPQ, CFB, CHRNA1, COL20A1, COL4A6, DPP4, FCGR3A, H1F0, H2AFX, H3F3B, HBA1, HBA2, HBD, HBM, HLA-C, HLA-DQB1, HLA-F, HSPB7, IFNG, IGFL2, IGH2, IGHV7-81, IL1RAPL2, IL6R, ITGB2, keratin 73, KRT19, KRTAP9-3, KRTAP9-8, MBP, MLF1IP, MYT1L, NOLA3, POLR2H, POLR2I, POLR3D, POLR3H, PTBP1, STK19, and UEVLD, or fragments thereof.

19. A method of predicting a likelihood of a patient having a disease, comprising:
determining autoantibody reactivity against one or more antigens, or their variants, in a sera sample obtained from a patient;
predicting a likelihood of a disease from reference samples derived from sera of patients diagnosed as having the disease, wherein an increased or decreased autoantibody reactivity against the one or more antigens is positively correlated with increased likelihood of the disease in the patient; and
wherein the one or more antigens are selected from a group consisting of BRCA1, CD88, CSF2RA, HBZ, HSPD1, IFNA7, IL12A, IL17D, KRT17, KRT18, KRT24, KRT5, MYL6, MYO9B, PARP12, PECAM1, POLR2I, POLR3GL, SC65, SLC5A5, UTP14a, DPP4, IL6R, ITGB2, MLF1IP, MYO1A, POLR2H, CD1D, IRF8, ITGA2B, MYO7B, PSG1, PTBP1, CD1C, CD46, CENPQ, CFB, HLA-DQB1, KRTAP9-3, MYT1L, SLC7A5, TPO, CD14, CD55, COL9A1, COLQ, DLAT, FGF7, H3F3B, IL1RAPL2, IL8, PADI4, PIP4K2C, PLAUR, STK19, APOH, BANK1, BLK, CD3E, CD70, CD80, CD86, CEACAM6, CEACAM8, CENPT, COL1A2, DDC, FCGR1A, H2AFX, H2AFY, HBA1, HBA2, HBD, HSP90B1, HSPB7, IGHG2, IGHG4, IGHM, IGHV4-31, IL6, ITGB3BP, KRTAP13-1, MBP, MOBP, MS4A8B, MYH9, MYO1D, NMNAT2, NOL1, PDCD1, POLR2C, POLR2J2, POLR3D, PSIP1, SRP19, STAT4, CALR3, CD34, CD69, CD93, CENPA, CHRNA1, COL20A1, COL4A6, FCGR3A, H1F0, HBM, HLA-C, HLA-F, IFNG, IGFL2, IGH2, IGHV7-81, KRT73, KRT19, KRTAP9-8, NOLA3, POLR3H, and UEVLD, or fragments thereof.

20. The method of claim 19, further comprising:
assaying the reactivity of autoantibodies in the sera sample;
normalizing the level of the autoantibodies reactivity against a level of at least one reference autoantibody reactivity in the sera sample to provide a normalized reactivity level; and
comparing the normalized reactivity level to reactivity levels obtained from the reference samples derived from diseased patients;
wherein increased normalized reactivity levels against the one or more antigens positively correlates to the increased likelihood of the disease in the patient.
